(19) United States
(12) Patent Application Publication
(10) Pub. No.: US 2024/0052327 A1
(43) Pub. Date: Feb. 15, 2024

(54) METHOD OF CONTROLLING TOTAL SIALIC ACID CONTENT (TSAC) DURING MANUFACTURING OF ALKALINE PHOSPHATASE

(71) Applicant: Alexion Pharmaceuticals, Inc., Boston, MA (US)

(72) Inventors: Meghan DeWitt, Madison, CT (US); Siguang Sui, South Glastonbury, CT (US); Rahul Godawat, Woodbridge, CT (US); Sarah Berendes, Meriden, CT (US)

(73) Assignee: Alexion Pharmaceuticals, Inc., Boston, MA (US)

(21) Appl. No.: 18/032,973

(22) PCT Filed: Oct. 21, 2021
(86) PCT No.: PCT/US2021/055991

§ 371 Date: Apr. 20, 2023

Related U.S. Application Data
(60) Provisional application No. 63/105,052, filed on Oct. 23, 2020.

(51) Int. Cl. C12N 9/16

(52) U.S. Cl. CPC C12N 9/16; C12Y 301/03001; C07K 2319/30

(57) ABSTRACT

Featured are methods of manufacturing recombinant alkaline phosphatases, such as asfotase alfa, that provide more precise quality control over total sialic acid content (TSAC) concentration in the final product by measuring TSAC concentration during fermentation and adjusting downstream production steps in response.

### CROSS-REFERENCE TO RELATED APPLICATIONS

This application claims the benefit of U.S. Provisional Application No. 63/105,052, filed Oct. 23, 2020, the contents of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Oct. 19, 2021 is named 0608WO_SL.txt and is 15,286 bytes in size.

### BACKGROUND

Hypophosphatasia (HPP) is a life-threatening, genetic, and ultra-rare metabolic disorder that results in a failure to produce functional tissue nonspecific alkaline phosphatase (TNSALP). It leads to the accumulation of unmineralized bone matrix (e.g., rickets, osteomalacia), characterized by hypo-mineralization of bones and teeth. When growing bone does not mineralize properly, impairment of growth disfigures joints and bones. This result in turn impacts motor performance, respiratory function, and may even lead to death. HPP includes perinatal, infantile, juvenile (or childhood), and adult HPP. Historically, six clinical forms were defined, most based upon age at symptom onset, including perinatal, benign prenatal, infantile, juvenile, adult, and odonto-HPP.

Asfotase alfa (STRENSIQ®, Alexion Pharmaceuticals, Inc.) is an approved, first-in-class targeted enzyme replacement therapy designed to address defective endogenous TNSALP levels. Asfotase alfa is a soluble fusion glycoprotein comprised of the catalytic domain of human TNSALP, a human immunoglobulin G1 Fc domain, and a deca-aspartate peptide (e.g., D10) (SEQ ID NO: 2) used as a bone-targeting domain. In vitro, asfotase alfa binds with a greater affinity to hydroxyapatite than does soluble TNSALP lacking the deca-aspartate peptide, thus allowing the TNSALP moiety of asfotase alfa to efficiently degrade excess local inorganic pyrophosphate (PPi) and restore normal mineralization to bones. Pyrophosphate hydrolysis promotes bone mineralization, and its effects were similar among the species evaluated in nonclinical studies.

Asfotase alfa is a eukaryotic protein that contains post-translation modifications, e.g., glycosylation (e.g., sialyation). Production of commercial scale quantities of therapeutically effective alkaline phosphatases such as asfotase alfa involves a multi-step manufacturing process, the conditions of which can significantly affect the final product. During this process, post-translationally modified products can be exposed to glycosidases or other hydrolytic conditions during one or more steps of the manufacturing process that negatively impact the post-translational modifications. For example, added sialic acid moieties may be lost. These changes can reduce the half-life and enzymatic activity of large quantities of the batch product. Accordingly, improved methods of manufacturing alkaline phosphatases are needed to improve quality control of the final protein product and its glycosylation characteristics.

### BRIEF SUMMARY OF THE DISCLOSURE

Disclosed herein are manufacturing processes that can be used to improve quality control of glycosylation in the production of alkaline phosphatases (e.g., asfotase alfa). The methods can also be used for maintaining, preserving, modulating, and/or improving the enzymatic activity, and particularly maintaining, controlling, and/or improving half-life of a recombinant protein, such as an alkaline phosphatase (e.g., asfotase alfa) produced by cultured mammalian cells, particularly by cultured Chinese Hamster Ovary (CHO) cells. Such alkaline phosphatases (e.g., asfotase alfa) are suitable for use in therapy, for example, for treatment of conditions associated with decreased alkaline phosphatase protein levels (e.g., HPP) and/or function (e.g., insufficient cleavage of inorganic pyrophosphate (PPi), etc.) in a subject, for example, a human subject.

In one aspect, featured is a method of producing a recombinant alkaline phosphatase. The method includes the steps of: inoculating a bioreactor with a cell (e.g., a mammalian cell, e.g., Chinese Hamster Ovary (CHO) cell) expressing a recombinant alkaline phosphatase; obtaining an aqueous culture medium that includes the recombinant alkaline phosphatase; obtaining an aliquot from the aqueous culture medium at from about day 6 to about day 10, particularly from about day 6 to about day 8 (e.g., about day 6, about day 7, about day 8, about day 9, about day 10, e.g., about day 7) after inoculation; quantifying the total sialic acid content (TSAC) molar concentration per mole of the recombinant alkaline phosphatase in the aliquot; harvesting the aqueous culture medium; and performing a filtration step (e.g., ultrafiltration, diafiltration, or a combination thereof); ultimately to obtain a bulk drug substance (BDS). The method may further include additional downstream purification steps between the filtration step and obtaining the BDS (FIG. 1).

The aliquot of the culture medium from the fermentation stage is used to determine the amount of time during which the filtration pool is held. For example, if the aliquot has a TSAC concentration of less than about 2.5 mol/mol, then the filtration step may be held for less than about nine hours. If the aliquot has a TSAC concentration of from about 2.5 mol/mol to about 2.7 mol/mol, then the filtration step may be held for from about 10 hours to about 14 hours. If the aliquot has a TSAC concentration of from about 2.8 mol/mol to about 3.0 mol/mol, then the filtration step may be held for from about 23 hours to about 27 hours. If the aliquot has a TSAC concentration of greater than about 3.0 mol/mol, then the filtration step may be held for from about 38 hours to about 42 hours.

In one embodiment, if the aliquot has a TSAC concentration of less than about 2.5 mol/mol, then the filtration step may be held for about 7+/−2 hours or less (e.g., between about 5-9 hours). If the aliquot has a TSAC concentration of from about 2.5 mol/mol to about 2.7 mol/mol, then the filtration step may be held for about 18+/−2 hours or less (e.g., between about 16-20 hours). If the aliquot has a TSAC concentration of greater than about 2.7 mol/mol, then the filtration step may be held for about 32+/−2 hours or less (e.g., between about 30-34 hours).

In another embodiment, if the aliquot has a TSAC concentration of less than or equal to about 2.3 mol/mol, then the filtration step may be held for about 18+/−4 hours or less (e.g., between about 14-22 hours). If the aliquot has a TSAC concentration of from greater than about 2.3 mol/mol to about 3.1 mol/mol (e.g., from about 2.4 mol/mol to about 3.1 mol/mol), then the filtration step may be held for about 32+/−4 hours or less (e.g., between about 28-36 hours). If the aliquot has a TSAC concentration of greater than about 3.1 mol/mol (e.g., greater than or equal to about 3.2 mol/mol), then the filtration step may be held for about 44+/−4 hours or less (e.g., between about 40-48 hours).

In another embodiment, if the aliquot has a TSAC concentration of less than about 2.4 mol/mol, then the filtration step may be held for about 17+/−3 hours or less (e.g., between about 14-20 hours). If the aliquot has a TSAC concentration of from about 2.4 mol/mol to about 3.6 mol/mol, then the filtration step may be held for about 31+/−3 hours or less (e.g., between about 28-34 hours). If the aliquot has a TSAC concentration of greater than about 3.6 mol/mol, then the filtration step may be held for about 45+/−3 hours (e.g., between about 42-48 hours). The alkaline phosphatase concentration during the filtration step may be from about 3.7+/−0.4 g/L.

The alkaline phosphatase concentration during the filtration step may be from about 1.8 g/L to about 5.0 g/L (e.g., from about 1.8 to about 4.3 g/L, e.g., about 2.3 g/L, about 3.1 g/L, about 3.7 g/L). The TSAC concentration of the BDS may be from about 1.2 mol/mol to about 3.0 mol/mol (e.g., from about 1.6 mol/mol to about 2.4 mol/mol).

The filtration step may be held at a constant temperature, wherein the constant temperature is any temperature between a defined range. For example, the temperature may be held at from about 15° C. to about 25° C. (e.g., from about 19° C. to about 25° C., e.g., about 22° C.).

The aliquot may be obtained aseptically from the bioreactor in order to prevent contamination. The aliquot may be from about 1 mL to about 1000 mL (e.g., from about 25 mL to about 500 mL, e.g., from about 50 mL to about 300 mL, e.g., about 100 mL or about 200 mL).

Obtaining the aliquot may further include centrifuging the aliquot, and, optionally, removing the supernatant from the aliquot. This step may also include purifying the alkaline phosphatase from the supernatant using a chromatography column (e.g., a Protein A column, e.g., a 1 mL HiTrap Protein A column; 600 μl Protein A Robocolumn; or MabSelect Sure Protein A solid phase column). In some embodiments, the alkaline phosphatase may be subject to a buffer exchange. The alkaline phosphatase may also be concentrated, e.g., before determining TSAC analysis. The TSAC analysis, which includes quantifying TSAC concentration, may include performing acid hydrolysis to release the TSAC.

Following purification of the alkaline phosphatase, the alkaline phosphatase may be lyophilized and/or placed into a vial.

The bioreactor may be any suitable size, e.g., for commercial scale production of the alkaline phosphatase. For example, the bioreactor may have a volume of at least 2 L, at least 10 L, at least 1,000 L, at least 10,000 L, or at least 20,000 L. The volume may be about 10,000 L or about 20,000 L.

Any suitable cell culture medium may be used, such as serum-free medium. Some examples that are suitable include, for example, EX-CELL® 302 Serum-Free Medium; CD DG44 Medium; BD SELECT™ Medium; SFM4CHO Medium; and combinations thereof.

The alkaline phosphatase may include the structure of W-sALP-X-Fc-Y-Dn-Z, wherein:
W is absent or is an amino acid sequence of at least one amino acid;
X is absent or is an amino acid sequence of at least one amino acid;
Y is absent or is an amino acid sequence of at least one amino acid;
Z is absent or is an amino acid sequence of at least one amino acid;
Fc is a fragment crystallizable region;
Dn is a poly-aspartate, poly-glutamate, or combination thereof, wherein n=10 or 16; and
sALP is a soluble alkaline phosphatase.

In some embodiments, the recombinant alkaline phosphatase includes an amino acid sequence having at least 90% (e.g., at least 95%, 97%, 98%, or 99%) sequence identity to the sequence set forth in SEQ ID NO: 1. For example, the recombinant alkaline phosphatase may include or consist of the amino acid sequence set forth in SEQ ID NO: 1.

### Definitions

As used herein, the terms “about” and “approximately”, as applied to one or more particular cell culture conditions or numerical values, refer to a range of values that are +/−10% of a subject value.

The term “amino acid,” as used herein, refers to any of the twenty naturally occurring amino acids that are normally used in the formation of polypeptides, or analogs or derivatives of those amino acids. Amino acids of the present disclosure can be provided in medium to cell cultures. The amino acids provided in the medium may be provided as salts or in hydrate form.

The term “batch culture,” as used herein, refers to a method of culturing cells in which all of the components that will ultimately be used in culturing the cells, including the medium (see definition of “medium” below) as well as the cells themselves, are provided at the beginning of the culturing process. A batch culture is typically stopped at some point and the cells and/or components in the medium are harvested and optionally purified. In some embodiments, the methods described here are used in a batch culture.

The term “bioreactor” as used herein refers to any vessel used for the growth of a cell culture (e.g., a mammalian cell culture). The bioreactor can be of any size so long as it is useful for the culturing of cells. Typically, the bioreactor will be at least 1 liter and may be 10, 100, 250, 500, 1000, 2500, 5000, 8000, 10,000, 12,0000, 20,000, 22,000, 25,000, 30,000 liters or more, or any volume in between. In some embodiments, the bioreactor is 100 liters to 30,000 liters, 500 liters to 22,000 liters, 1,000 liters to 22,000 liters, 2,000 liters to 22,000 liters, 5,000 liters to 22,000 liters, or 10,000 liters to 22,000 liters. The maximum working volume of the bioreactor may vary by about 1% to 5%, e.g., may go up to about 22,250 liters or 33,000 liters. The internal conditions of the bioreactor, including, but not limited to pH and temperature, are typically controlled during the culturing period. The bioreactor can be composed of any material that is suitable for holding mammalian or other cell cultures suspended in media under the culture conditions of the present disclosure, including glass, plastic or metal. The term “production bioreactor” as used herein refers to the final bioreactor used in the production of the polypeptide or protein of interest. The volume of the large-scale cell culture production bioreactor is typically at least 500 liters and may be 1000, 2500, 5000, 8000, 10,000, 12,0000, 20,000 liters or more, or any volume in between. One of ordinary skill in the art will be aware of and will be able to choose suitable bioreactors for use in practicing the present disclosure.

The term “cell density,” as used herein, refers to the number of cells present in a given volume of medium.

The term “cell viability,” as used herein, refers to the ability of cells in culture to survive under a given set of culture conditions or experimental variations. The term as used herein also refers to that portion of cells which are alive at a particular time in relation to the total number of cells, living and dead, in the culture at that time.

The terms “culture” and “cell culture,” as used herein, refer to a cell population that is suspended in a medium (see definition of “medium” below) under conditions suitable for survival and/or growth of the cell population. As will be clear to those of ordinary skill in the art, these terms as used herein may refer to the combination comprising the cell population and the medium in which the population is suspended.

The term “fed-batch culture,” as used herein, refers to a method of culturing cells in which additional components are provided to the culture at some time subsequent to the beginning of the culture process. The provided components typically comprise nutritional supplements for the cells, which have been depleted during the culturing process. A fed-batch culture is typically stopped at some point and the cells and/or components in the medium are harvested and optionally purified. Fed-batch culture may be performed in the corresponding fed-batch bioreactor. In some embodiments, the method comprises a fed-batch culture.

The term “fragment,” as used herein, refers to a polypeptide and is defined as any discrete portion of a given polypeptide that is unique to or characteristic of that polypeptide. The term as used herein also refers to any discrete portion of a given polypeptide that retains at least a fraction of the activity of the full-length polypeptide. In some embodiments, the fraction of activity retained is at least 10% of the activity of the full-length polypeptide. In various embodiments, the fraction of activity retained is at least 20%, 30%, 40%, 50%, 60%, 70%, 80%, or 90% of the activity of the full-length polypeptide. In other embodiments, the fraction of activity retained is at least 95%, 96%, 97%, 98%, or 99% of the activity of the full-length polypeptide. In one embodiment, the fraction of activity retained is 100% of the activity of the full-length polypeptide. The term as used herein also refers to any portion of a given polypeptide that includes at least an established sequence element found in the full-length polypeptide. In some embodiments, the sequence element spans at least 4-5 amino acids of the full-length polypeptide. In some embodiments, the sequence element spans at least about 10, 15, 20, 25, 30, 35, 40, 45, 50 or more amino acids of the full-length polypeptide.

The terms “glycoprotein” or “glycoproteins,” as used herein, refer to a protein or polypeptide with carbohydrate groups (such as sialic acid) attached to the polypeptide chain.

The terms “medium”, “media”, “cell culture medium”, and “culture medium,” as used herein, refer to a solution containing nutrients which nourish growing mammalian cells. Typically, these solutions provide essential and non-essential amino acids, vitamins, energy sources, lipids, and trace elements required by the cell for minimal growth and/or survival. The solution may also contain components that enhance growth and/or survival above the minimal rate, including hormones and growth factors. The solution may be, e.g., formulated to a pH and salt concentration optimal for cell survival and proliferation. In some embodiments, a culture medium may be a “defined media”—a serum-free media that contains no proteins, hydrolysates or components of unknown composition. Defined media are free of animal-derived components and all components have a known chemical structure. In some embodiments, the culture medium is a basal medium, e.g., an undefined medium containing a carbon source, water, salts, a source of amino acids and nitrogen (e.g., animal, e.g., beef, or yeast extracts). Various mediums are commercially available and are known to those in the art. In some embodiments, the culture medium is selected from EX-CELL® 302 Serum-Free Medium (Sigma Aldrich, St. Louis, MO), CD DG44 Medium (ThermoFisher Scientific, Waltham, MA), BD Select Medium (BD Biosciences, San Jose, CA), or a mixture thereof, or a mixture of BD Select Medium with SFM4CHO Medium (HYCLONE™, Logan UT). In some embodiments, the culture medium comprises a combination of SFM4CHO Medium and BD SELECT™ Medium. In some embodiments, the culture medium comprises a combination of SFM4CHO Medium and BD SELECT™ Medium at a ratio selected from 90/10, 80/20, 75/25, 70/30, 60/40, or 50/50, which includes any intermediate ratio therebetween. In some embodiments, the culture medium comprises a combination of SFM4CHO Medium and BD SELECT™ Medium at a ratio of 70/30 to 90/10. In some embodiments, the culture medium comprises a combination of SFM4CHO Medium and BD SELECT™ Medium at a ratio 75/25. EX-CELL® 302 Serum-Free Medium contains 0.1% PLURONIC® F68, 3.42 g/L glucose, 7.5 mM HEPES, and 1.6 g/L sodium bicarbonate. BD SELECT™ Medium contains human recombinant insulin, hypoxanthine, thymidine, and low endotoxin (55.0 EU/mL), at pH 7.1+/−0.2. CD DG44 Medium is a chemically defined, protein-free, hydrolysate-free medium that contains hypoxanthine and thymidine and L-glutamine without PLURONIC® F-68. In some embodiments, alkaline phosphatase (e.g., asfotase alfa) is produced by a process in which extra boluses of culture medium are added to the production bioreactor. For example, one, two, three, four, five, six, or more boluses of culture medium may be added. In one particular embodiment, three boluses of culture medium are added. In various embodiments, such extra boluses of culture medium may be added in various amounts. For example, such boluses of culture medium may be added in an amount of about 20%, 25%, 30%, 33%, 40%, 45%, 50%, 60%, 67%, 70%, 75%, 80%, 90%, 100%, 110%, 120%, 125%, 130%, 133%, 140%, 150%, 160%, 167%, 170%, 175%, 180%, 190%, 200%, or more, of the original volume of culture medium in the production bioreactor. In one particular embodiment, such boluses of culture medium may be added in an amount of about 33%, 67%, 100%, or 133% of the original volume. In various embodiments, such addition of extra boluses may occur at various times during the cell growth or protein production period. For example, boluses may be added at day 1, day 2, day 3, day 4, day 5, day 6, day 7, day 8, day 9, day 10, day 11, day 12, or later in the process. In one particular embodiment, such boluses of culture medium may be added in every other day (e.g., at (1) day 3, day 5, and day 7; (2) day 4, day 6, and day 8; or (3) day 5, day 7, and day 9. In practice, the frequency, amount, time point, and other parameters of bolus supplements of culture medium may be combined freely according to the above limitation and determined by experimental practice.

The terms “Osmolality” and “osmolarity,” as used herein, refer to a measure of the osmotic pressure of dissolved solute particles in an aqueous solution. The solute particles include both ions and non-ionized molecules. Osmolality is expressed as the concentration of osmotically active particles (e.g., osmoles) dissolved in 1 kg of solution (1 mOsm/kg H2O at 38° C. is equivalent to an osmotic pressure of 19 mm Hg). “Osmolarity,” by contrast, refers to the number of solute particles dissolved in 1 liter of solution. When used herein the abbreviation “mOsm” means “milliosmoles/kg solution”.

The term “perfusion culture,” as used herein, refers to a method of culturing cells in which additional components are provided continuously or semi-continuously to the culture subsequent to the beginning of the culture process. The provided components typically comprise nutritional supplements for the cells, which have been depleted during the culturing process. A portion of the cells and/or components in the medium are typically harvested on a continuous or semi-continuous basis and are optionally purified. In some embodiments, the nutritional supplements as described herein are added in a perfusion culture, e.g., they are provided continuously over a defined period of time.

The term “polypeptide,” as used herein, refers a sequential chain of amino acids linked together via peptide bonds. The term is used to refer to an amino acid chain of any length, but one of ordinary skill in the art will understand that the term is not limited to lengthy chains and can refer to a minimal length chain comprising two amino acids linked together via a peptide bond.

The term “protein,” as used herein, refers to one or more polypeptides that function as a discrete unit. If a single polypeptide is the discrete functioning unit and does not require permanent physical association with other polypeptides in order to form the discrete functioning unit, the terms “polypeptide” and “protein” as used herein are used interchangeably.

The terms “recombinantly-expressed polypeptide” and “recombinant polypeptide,” as used herein, refer to a polypeptide expressed from a host cell that has been genetically engineered to express that polypeptide. The recombinantly-expressed polypeptide can be identical or similar to a polypeptide that is normally expressed in the mammalian host cell. The recombinantly-expressed polypeptide can also be foreign to the host cell, e.g., heterologous to peptides normally expressed in the host cell. Alternatively, the recombinantly-expressed polypeptide can be chimeric in that portions of the polypeptide contain amino acid sequences that are identical or similar to polypeptides normally expressed in the mammalian host cell, while other portions are foreign to the host cell.

The term “seeding,” as used herein, refers to the process of providing a cell culture to a bioreactor or another vessel. The cells may have been propagated previously in another bioreactor or vessel. Alternatively, the cells may have been frozen and thawed immediately prior to providing them to the bioreactor or vessel. The term refers to any number of cells, including a single cell. In various embodiments, alkaline phosphatase (e.g., asfotase alfa) is produced by a process in which cells are seeded in a density of about 1.0×10⁵ cells/mL, 1.5×10⁵ cells/mL, 2.0×10⁵ cells/mL, 2.5×10⁵ cells/mL, 3.0×10⁵ cells/mL, 3.5×10⁵ cells/mL, 4.0×10⁵ cells/mL, 4.5×10⁵ cells/mL, 5.0×10⁵ cells/mL, 5.5×10⁵ cells/mL, 6.0×10⁵ cells/mL, 6.5×10⁵ cells/mL, 7.0×10⁵ cells/mL, 7.5×10⁵ cells/mL, 8.0×10⁵ cells/mL, 8.5×10⁵ cells/mL, 9.0×10⁵ cells/mL, 9.5×10⁵ cells/mL, 1.0×10⁶ cells/mL, 1.5×10⁶ cells/mL, 2.0×10⁶ cells/mL, or a higher density. In one particular embodiment, in such process cells are seeded in a density of about 4.0×10⁵ cells/mL, 5.5×10⁵ cells/mL or 8.0×10⁵ cells/mL.

The terms “total Sialic Acid Content” or “TSAC,” as used herein, refer to the amount of sialic acid (a carbohydrate) on a particular protein molecule. It is expressed as moles TSAC per mole of protein, or, “mol/mol.” TSAC concentration is measured during the purification process. For example, one method of TSAC quantitation is where TSAC is released from asfotase alfa using acid hydrolysis, and the released TSAC is subsequently detected via electrochemical detection using high-performance anion-exchange chromatography with pulsed amperometric detection technique (“HPAE-PAD”).

The term “titer,” as used herein, refers to the total amount of recombinantly-expressed polypeptide or protein produced by a cell culture divided by a given amount of medium volume. Titer is typically expressed in units of milligrams of polypeptide or protein per milliliter of medium.

Acronyms used herein include, e.g., HCCF: Harvest Clarified Culture Fluid; UF: ultrafiltration, DF: diafiltration; VCD: Viable Cell Density; IVCC: Integral of Viable Cell Concentration; TSAC: Total Sialic Acid Content; HPAE-PAD: High-Performance Anion Exchange Chromatography with Pulsed Amperometric Detection; SEC: Size Exclusion Chromatography; AEX: Anion Exchange Chromatography; LoC: Lab-on-Chip; and MALDI-TOF: Matrix Assisted Laser Desorption/Ionization—Time of Flight.

As used herein, the term “hydrophobic interaction chromatography (HIC) column” refers to a column containing a stationary phase or resin and a mobile or solution phase in which the hydrophobic interaction between a protein and hydrophobic groups on the stationary phase or resin separates a protein from impurities including fragments and aggregates of the subject protein, other proteins or protein fragments and other contaminants such as cell debris, or residual impurities from other purification steps. The stationary phase or resin comprises a base matrix or support such as a cross-linked agarose, silica or synthetic copolymer material to which hydrophobic ligands are attached. Examples of such stationary phase or resins include phenyl-, butyl-, octyl-, hexyl- and other alkyl substituted agarose, silica, or other synthetic polymers. Columns may be of any size containing the stationary phase, or may be open and batch processed. In some embodiments, the recombinant alkaline phosphatase is isolated from the cell culture using HIC.

As used herein, the term “preparation” refers to a solution comprising a protein of interest (e.g., a recombinant alkaline phosphatase described herein) and at least one impurity from a cell culture producing such protein of interest and/or a solution used to extract, concentrate, and/or purify such protein of interest from the cell culture. For example, a preparation of a protein of interest (e.g., a recombinant alkaline phosphatase described herein) may be prepared by homogenizing cells, which grow in a cell culture and produce such protein of interest, in a homogenizing solution. In some embodiments, the preparation is then subjected to one or more purification/isolation process, e.g., a chromatography step.

As used herein, the term “solution” refers to a homogeneous, molecular mixture of two or more substances in a liquid form. Specifically, in some embodiments, the proteins to be purified, such as the recombinant alkaline phosphatases or their fusion proteins (e.g., asfotase alfa) in the present disclosure, represent one substance in a solution. The term “buffer” or “buffered solution” refers to solutions which resist changes in pH by the action of its conjugate acid-base range. Examples of buffers that control pH at ranges of about pH 5 to about pH 7 include HEPES, citrate, phosphate, acetate, and other mineral acids or organic acid buffers, and combinations of these. Salt cations include sodium, ammonium, and potassium. As used herein the term “loading buffer/solution” or “equilibrium buffer/solution” refers to the buffer/solution containing the salt or salts which is mixed with the protein preparation for loading the protein preparation onto a chromatography column, e.g., HIC column. This buffer/solution is also used to equilibrate the column before loading, and to wash to column after loading the protein. The “elution buffer/solution” refers to the buffer/solution used to elute the protein from the column. As used herein, the term “solution” refers to either a buffered or a non-buffered solution, including water.

The term “sialic acid” refers generally to N- or O-substituted derivatives of neuraminic acid, a monosaccharide with a nine-carbon backbone. Sialic acid may also refer specifically to the compound N-acetylneuraminic acid and is sometimes abbreviated as Neu5Ac or NANA. Presence of sialic acid may affect absorption, serum half-life, and clearance of glycoproteins from the serum, as well as physical, chemical, and immunogenic properties of the glycoprotein. In some embodiments of the present disclosure, sialic acid associated with alkaline phosphatases, e.g., asfotase alfa, impacts the half-life of the molecule in physiological conditions. In some embodiments, precise and predictable control of total sialic acid content (TSAC) of asfotase alfa serves as a critical quality attribute for recombinant asfotase alfa. In some embodiments, the TSAC is 1.2 to 3.0 mol/mol asfotase alfa monomer. In some embodiments, TSAC is generated in the recombinant protein production process in the bioreactor. In some embodiments, the disclosure provides a method of controlling total sialic acid content (TSAC) in a TSAC-containing recombinant protein through mammalian cell culture, comprising at least one purification step and at least one chromatography step. In some embodiments, the purification and chromatography steps lead to decreased glycosidase activity, and thus increased total sialic acid content of the recombinant protein.

The term “sialylation” refers to a specific type of glycosylation, e.g., the addition of one or more sialic acid molecules to biomolecules, particularly, the addition of one or more sialic acid molecules to proteins. In some embodiments of the present disclosure, sialylation is performed by a sialyltransferase enzyme. In some embodiments, sialyltransferases add sialic acid to nascent oligosaccharides and/or to N- or O-linked sugar chains of glycoproteins. In some embodiments, sialyltransferases are present natively in the cells producing recombinant alkaline phosphatase. In some embodiments, sialyltransferases are present in the cell culture medium and/or nutrient supplement used in culturing the cells producing recombinant alkaline phosphatase. In some embodiments, sialyltransferases are produced recombinantly, using recombinant protein expression methods known in the art. In some embodiments, recombinant sialyltransferases produced separately from the recombinant alkaline phosphatases are added exogenously to the cell culture, the harvest clarified culture fluid (HCCF), and/or the filtration pool.

In some embodiments of the present disclosure, sialic acid groups are removed from glycoproteins (e.g., “desialylation”) by hydrolysis. In some embodiments, desialylation is performed by a glycosidase enzyme. As used herein, “glycosidase,” also called “glycoside hydrolase,” is an enzyme that catalyzes the hydrolysis of a bond joining a sugar of a glycoside to an alcohol or another sugar unit. Examples of glycosidases include amylase, xylanase, cellulase, and sialidase. In some embodiments, desialylation is performed by a sialidase enzyme. In some embodiments, sialidases hydrolyze glycosidic linkages of terminal sialic acid residues in glycoproteins, glycolipids, oligosaccharides, colominic acid, and/or synthetic substrates. In some embodiments, sialidases are present in the cell culture medium producing recombinant alkaline phosphatase. In some embodiments, sialidase activity is dependent on and/or correlates with total protein concentration. In some embodiments, sialidases are essentially inactive until a critically high protein concentration, at which point the sialidase is activated. In some embodiments, sialidases are present in the HCCF or the filtration pool of the cell culture producing recombinant alkaline phosphatase. In some embodiments, sialidases remove sialic acid moieties from glycosylation sites on recombinant alkaline phosphatase, e.g., asfotase alfa, effectively reducing the TSAC of the recombinant alkaline phosphatase. In some embodiments, sialidases are selectively removed from the cell culture, the HCCF, and/or the filtration pool. Sialidases can be selectively removed by, e.g., one or a combination of sialidase-specific inhibitors, antibodies, ion exchange and/or affinity chromatography, immunoprecipitation, and the like. For an overview of how bioprocess conditions affect the sialic acid content of proteins, see Gramer et al., Biotechnol. Prog. 9(4):366-373 (1993), the disclosure of which is hereby incorporated by reference in its entirety. In some embodiments, the present disclosure provides a method of controlling glycosidase activity in mammalian cell culture producing recombinant protein, comprising at least one purification and at least one chromatography step. In some embodiments, the purification and chromatography steps lead to decreased glycosidase activity, and thus increased total sialic acid content of the recombinant protein.

The term “harvest clarified culture fluid,” abbreviated as HCCF, refers to a clarified, filtered fluid harvested from a cell culture, e.g., a cell culture in a bioreactor. The HCCF is typically free of cells and cellular debris (such as, e.g., insoluble biomolecules) which may be present in the cell culture. In some embodiments of the present disclosure, HCCF is generated through centrifugation, depth filtration, sterile filtration, and/or chromatography. In some embodiments, a cell culture fluid from the bioreactor is first centrifuged and/or filtered, then subjected to at least one chromatography step in order to generate the HCCF. In some embodiments, the HCCF is concentrated prior to and/or after the at least one chromatography step. In some embodiments, the HCCF is diluted after the at least one chromatography step. In some embodiments, the HCCF from the cell culture producing recombinant alkaline phosphatase contains the recombinant alkaline phosphatase and contaminant proteins. In some embodiments, the contaminant proteins in the HCCF include sialidase enzymes.

The terms “filtration” and “flow filtration” refer to a pressure driven process that uses membranes to separate components in a liquid solution or suspension based on their size and charge differences. Flow filtration may be normal flow filtration or “tangential flow filtration,” also known as TFF or cross-flow filtration. TFF is typically used for clarifying, concentrating, and purifying proteins. During a TFF process, fluid is pumped tangentially along the surface of at least one membrane. An applied pressure serves to force a portion of the fluid through the membrane to the downstream side as “filtrate.” Particulates and macromolecules that are too large to pass through the membrane pores are retained on the upstream side as “retentate.” TFF may be used in various forms, including, for example, microfiltration, ultrafiltration—which includes virus filtration and high performance TFF, reverse osmosis, nanofiltration, and diafiltration. In some embodiments of the present disclosure, one or more of the TFF forms are used in combination for protein processing and/or purification. In some embodiments, ultrafiltration and diafiltration are used in combination for purifying a recombinant alkaline phosphatase. Ultrafiltration and diafiltration are described herein.

“Ultrafiltration,” or “UF,” is a purification process used to separate proteins from buffer components for buffer exchange, desalting, or concentration. Depending on the protein to be retained, membrane molecular weight limits in the range of about 1 kD to about 1000 kD are used. In some embodiments, UF is a TFF process.

“Diafiltration,” or “DF,” is a purification process that washes smaller molecules through a membrane and leaves larger molecules in the retentate without ultimately changing concentration. Typically, DF is used in combination with another purification processes to enhance product yield and/or purity. During DF, solution (e.g., water or buffer) is introduced into the sample reservoir while filtrate is removed from the unit operation. In processes where the desired product is in the retentate, diafiltration washes components out of the product pool into the filtrate, thereby exchanging buffers and reducing the concentration of undesirable species. When the product is in the filtrate, diafiltration washes it through the membrane into a collection vessel. In some embodiments, DF is a TFF process.

The term “filtration pool,” sometimes also referred to as the “UFDF pool” or the “UFDF,” refers to a total volume of fluid from a filtration process, typically from a combined ultrafiltration/diafiltration (UF/DF) process. In the context of protein purification, the UFDF refers to the retentate from an ultrafiltration/diafiltration process.

### BRIEF DESCRIPTION OF THE DRAWINGS

FIG. 1 is a flowchart showing the production process of a recombinant alkaline phosphatase, such as asfotase alfa.

FIG. 2 is a graph showing TSAC content of the asfotase alfa at the harvest, protein A pool step and the final BDS for multiple batches.

FIG. 3 is a graph showing TSAC content of the asfotase alfa at Day 7 after inoculation, at the harvest step and the final BDS for multiple batches.

### DETAILED DESCRIPTION

The present disclosure provides improved methods of manufacturing recombinant glycoproteins, such as alkaline phosphatases (e.g., asfotase alfa), that provide improved quality control over total sialic acid content (TSAC) concentration in the final product by measuring TSAC concentration during fermentation and adjusting downstream production steps in response to the TSAC concentration measurements. The methods allow modulation of TSAC of the final product by using a dynamic control strategy to respond to potentially variable ranges of TSAC levels from bioreactor cell culture output. Ultimately, this method provides uniform properties of the recombinant alkaline phosphatase for a commercial production. The methods described herein provide a resulting product in which TSAC of the final product is tightly controlled over a range of input bioreactor aqueous culture medium TSAC levels.

### Methods of Manufacture

The methods described herein include the steps of: inoculating a bioreactor with a cell (e.g., a mammalian cell, e.g., Chinese Hamster Ovary (CHO) cell) expressing a recombinant alkaline phosphatase; obtaining an aqueous culture medium that includes the recombinant alkaline phosphatase; obtaining an aliquot from the aqueous culture medium at from about day 6 to about day 10, particularly from about day 6 to about day 8 (e.g., about day 6, about day 7, about day 8, about day 9, about day 10, e.g., about day 7) after inoculation; quantifying the total sialic acid content (TSAC) molar concentration per mole of the recombinant alkaline phosphatase in the aliquot; harvesting the aqueous culture medium; and performing a filtration step (e.g., ultrafiltration, diafiltration, or a combination thereof) to obtain a bulk drug solution (BDS).

During fermentation, the aliquot of the culture medium is used to determine the amount of time during which the filtration step is held. For example, if the aliquot has a TSAC concentration of less than about 2.5 mol/mol, then the filtration step may be held for less than about nine hours. If the aliquot has a TSAC concentration of from about 2.5 mol/mol to about 2.7 mol/mol, then the filtration step may be held for from about 10 hours to about 14 hours. If the aliquot has a TSAC concentration of from about 2.8 mol/mol to about 3.0 mol/mol, then the filtration step may be held for from about 23 hours to about 27 hours. If the aliquot has a TSAC concentration of greater than about 3.0 mol/mol, then the filtration step may be held for from about 38 hours to about 42 hours.

In one embodiment, if the aliquot has a TSAC concentration of less than or equal to about 2.3 mol/mol, then the filtration step may be held for about 18+/−4 hours. If the aliquot has a TSAC concentration of from about 2.4 mol/mol to about 3.1 mol/mol, then the filtration step may be held for about 32+/−4 hours. If the aliquot has a TSAC concentration of greater than or equal to about 3.2 mol/mol, then the filtration step may be held for about 44+/−4 hours.

In another alternative embodiment, if the aliquot has a TSAC concentration of less than about 2.4 mol/mol, then the filtration step may be held for about 17+/−3 hours. If the aliquot has a TSAC concentration of from about 2.4 mol/mol to about 3.6 mol/mol, then the filtration step may be held for about 31+/−3 hours. If the aliquot has a TSAC concentration of greater than about 3.6 mol/mol, then the filtration step may be held for about 45+/−3 hours.

The methods may produce a BDS in which the TSAC concentration is controlled to a range of from about 1.2 mol/mol to about 3.0 mol/mol (e.g., from about 1.6 mol/mol to about 2.4 mol/mol). This range of TSAC concentration provides a bulk sample of recombinant alkaline phosphatase in commercially relevant scales that is stable (e.g., therapeutically effective half-life) and enzymatically active for use in human patients.

The alkaline phosphatase protein described herein (e.g., asfotase alfa) may be produced by mammalian or other cells, particularly CHO cells, using methods known in the art. Such cells may be grown in culture dishes, flask glasses, or bioreactors. Specific processes for cell culture and producing recombinant proteins are known in the art, such as described in Nelson and Geyer, 1991 Bioprocess Technol. 13:112-143 and Rea et al., Supplement to BioPharm International March 2008, 20-25. Exemplary bioreactors include batch, fed-batch, and continuous reactors. In some embodiments, the alkaline phosphatase protein is produced in a fed-batch bioreactor.

Cell culture processes have variability caused by, for example, variable physicochemical environment, including but not limited to, changes in pH, temperature, temperature changes, timing of temperature changes, cell culture media composition, cell culture nutrient supplements, raw material lot-to-lot variation, medium filtration material, bioreactor scale difference, gassing strategy (air, oxygen, and carbon dioxide), etc. As disclosed herein, the yield, relative activity profile, and glycosylation profile of manufactured alkaline phosphatase protein may be affected and may be controlled within particular values by alterations in one or more of these parameters.

For recombinant protein production in cell culture, the recombinant gene with the necessary transcriptional regulatory elements is first transferred to a host cell by methods known in the biotechnological arts. Optionally, a second gene is transferred that confers to recipient cells a selective advantage. In the presence of the selection agent, which may be applied a few days after gene transfer, only those cells that express the selector gene survive. Two exemplary genes for such selection are dihydrofolate reductase (DHFR), an enzyme involved in nucleotide metabolism, and glutamine synthetase (GS). In both cases, selection occurs in the absence of the appropriate metabolite (hypoxanthine and thymidine, in the case of DHFR, and glutamine in the case of GS), preventing growth of any non-transformed cells. In general, for efficient expression of the recombinant protein, it is not important whether the biopharmaceutical-encoding gene and selector genes are on the same plasmid or not.

Following selection, surviving cells may be transferred as single cells to a second cultivation vessel, and the cultures are expanded to produce clonal populations. Eventually, individual clones are evaluated for recombinant protein expression, with the highest producers being retained for further cultivation and analysis. From these candidates, one cell line with the appropriate growth and productivity characteristics is chosen for production of the recombinant protein. A cultivation process is then developed that is determined by the production needs and the requirements of the final product.

### Cells

Any mammalian cell or non-mammalian cell type, which can be cultured to produce a polypeptide may be utilized in accordance with the present disclosure. Non-limiting examples of mammalian cells that may be used include, e.g., Chinese hamster ovary cells+/−DHFR (CHO, Urlaub and Chasin, 1980 Proc. Natl. Acad. Sci. USA, 77:4216); BALB/c mouse myeloma line (NSO/1, ECACC Accession No: 85110503); human retinoblasts (PER.C6 (CruCell, Leiden, The Netherlands)); monkey kidney CVI line transformed by SV40 (COS-7, ATCC CRL 1651); human embryonic kidney line (293 or 293 cells subcloned for growth in suspension culture, Graham et al., 1977 J. Gen Virol., 36:59); baby hamster kidney cells (BHK, ATCC CCL 10); mouse Sertoli cells (TM4, Mather, Biol. Reprod., 23:243-251 (1980)); monkey kidney cells (CVI ATCC CCL 70); African green monkey kidney cells (VERO-76, ATCC CRL-I 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; and a human hepatoma line (Hep G2). In a particular embodiment, culturing and expression of polypeptides and proteins occurs from a Chinese Hamster Ovary (CHO) cell line.

Additionally, any number of commercially and non-commercially available hybridoma cell lines that express polypeptides or proteins may be utilized in accordance with the present disclosure. One skilled in the art will appreciate that hybridoma cell lines might have different nutrition requirements and/or might require different culture conditions for optimal growth and polypeptide or protein expression and will be able to modify conditions as needed.

### Seeding Density

In the present disclosure, Chinese Hamster Ovary (CHO) cells are inoculated, e.g., seeded, into the culture medium. Various seeding densities can be used. In some embodiments, a seeding density of 1.0×10² cells/mL to 1.0×10⁹ cells/mL (e.g., 1.0×10³ cells/mL to 1.0×10⁸, e.g., 1.0×10⁴ cells/mL to 1.0×10⁷) can be used. In some embodiments, a seeding density of 1.0×10⁵ cells/mL to 1.0×10⁶ cells/mL can be used. In some embodiments, a seeding density of 4.0×10⁵ cells/mL to 8.0×10⁵ cells/mL can be used. In some embodiments, increased seeding density can impact fragmentation of asfotase alfa quality, as measured by SEC. In some embodiments, the seeding density is controlled when inoculating in order to reduce the risk of fragment generation.

### Temperature

Temperature may have an impact on several parameters including growth rate, aggregation, fragmentation, and TSAC. In some embodiments, the temperature remains constant when culturing the CHO cells in the culture medium. In some embodiments, the temperature is about 30° C. to about 40° C., or about 35° C. to about 40° C., or about 37° C. to about 39° C. when culturing the CHO cells in the culture medium. In some embodiments, the temperature is about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C., or about 40° C. when culturing the CHO cells in the culture medium. In some embodiments, the temperature is constant for 40 to 200 hours after inoculation. In some embodiments, the temperature is constant for 50 to 150 hours, or 60 to 140 hours, or 70 to 130 hours, or 80 to 120 hours, or 90 to 110 hours after inoculation. In some embodiments, the temperature is constant for 80 to 120 hours after inoculation. In some embodiments, the temperature is constant for 90 hours, 92 hours, 94 hours, 96 hours, 98 hours, 100 hours, 102 hours, 104 hours, 106 hours, 108 hours or 110 hours after inoculation.

### Temperature Shifting

Run times of cell culture processes, especially non-continuous processes (e.g., fed-batch processes in bioreactors), are usually limited by the remaining viability of the cells, which typically declines over the course of the run. Therefore, extending the length of time for cell viability is desired for improving recombination protein production. Product quality concerns also offer a motivation for minimizing decreases in viable cell density and maintaining high cell viability, as cell death can release sialidases to the culture supernatant, which may reduce the sialic acid content of the protein expressed. Protein purification concerns offer yet another motivation for minimizing decreases in viable cell density and maintaining high cell viability. Cell debris and the contents of dead cells in the culture can negatively impact one's ability to isolate and/or purify the protein product at the end of the culturing run. Thus, by keeping cells viable for a longer period of time in culture, there is a reduction in the contamination of the culture medium by cellular proteins and enzymes (e.g., cellular proteases and sialidases) that may cause degradation and ultimate reduction in the quality of the desired glycoprotein produced by the cells.

Many methods may be applied to achieve high cell viability in cell cultures. One involves lowering culture temperature following initial culturing at a normal temperature. For example, see Ressler et al., 1996, Enzyme and Microbial Technology 18:423-427). Generally, the mammalian or other types of cells capable of expressing a protein of interest are first grown under a normal temperature to increase cell numbers. Such “normal” temperatures for each cell type are generally around 37° C. (e.g., from about 35° C. to about 39° C., including, for example, 35.0° C., 35.5° C., 36.0° C., 36.5° C., 37.0° C., 37.5° C., 38.0° C., 38.5° C., and/or 39.0° C.). In one particular embodiment, the temperature for producing asfotase alfa is first set at about 37° C. When a reasonably high cell density is reached, the culturing temperature for the whole cell culture can then be shifted (e.g., decreased) to promote protein production. In most cases lowering temperature shifts the cells towards the non-growth G1 portion of the cell cycle, which may increase cell density and viability, as compared to the previous higher-temperature environment. In addition, a lower temperature may also promote recombinant protein production by increasing the cellular protein production rate, facilitating protein post-translational modification (e.g., glycosylation), decreasing fragmentation or aggregation of newly-produced proteins, facilitating protein folding and formation of 3D structure (thus maintaining activity), and/or decreasing degradation of newly produced proteins. In some embodiments, the temperature is decreased 3° C., 4° C., 5° C., 6° C., 7° C., 8° C., 9° C., or 10° C. In some embodiments, the temperature is decreased to about 27° C., 28° C., 29° C., 30° C., 31° C., 32° C., 33° C., 34° C., or 35° C. In some embodiments, the lower temperature is from about 30° C. to about 35° C. (e.g., 30.0° C., 30.5° C., 31.0° C., 31.5° C., 32.0° C., 32.5° C., 33.0° C., 33.5° C., 34.0° C., 34.5° C., and/or 35.0° C.). In other embodiments, the temperature for producing asfotase alfa is first set to from about 35.0° C. to about 39.0° C. and then shifted to from about 30.0° C. to about 35.0° C. In one embodiment, the temperature for producing asfotase alfa is first set at about 37.0° C. and then shifted to about 30° C. In another embodiment, the temperature for producing asfotase alfa is first set at about 36.5° C. and then shifted to about 33° C. In yet another embodiment, the temperature for producing asfotase alfa is first set at about 37.0° C. and then shifted to about 33° C. In yet a further embodiment, the temperature for producing asfotase alfa is first set at about 36.5° C. and then shifted to about 30° C. In other embodiments, multiple (e.g., more than one) steps of temperature shifting may be applied.

The time for maintaining the culture at a particular temperature prior to shifting to a different temperature may be determined to achieve a sufficient (or desired) cell density while maintaining cell viability and an ability to produce the protein of interest. In some embodiments, the cell culture is grown under the first temperature until the viable cell density reaches about 10⁵ cells/mL to about 10⁷ cells/mL (e.g., 1×10⁵, 1.5×10⁵, 2.0×10⁵, 2.5×10⁵, 3.0×10⁵, 3.5×10⁵, 4.0×10⁵, 4.5×10⁵, 5.0×10⁵, 5.5×10⁵, 6.0×10⁵, 6.5×10⁵, 7.0×10⁵, 7.5×10⁵, 8.0×10⁵, 8.5×10⁵, 9.0×10⁵, 9.5×10⁵, 1.0×10⁶, 1.5×10⁶, 2.0×10⁶, 2.5×10⁶, 3.0×10⁶, 3.5×10⁶, 4.0×10⁶4.5×10⁶, 5.0×10⁶ 5×10⁶, 6.0×10⁶, 6.5×10⁶, 7.0×10⁶, 7.5×10⁶, 8.0×10⁶, 8.5×10⁶, 9.0×10⁶, 9.5×10⁶, 1×10⁷ cell/mL, or more) before shifting to a different temperature. In one embodiment, the cell culture is grown under the first temperature until the viable cell density reaches about 2.5 to about 3.4×10⁶ cells/mL before shifting to a different temperature. In another embodiment, the cell culture is grown under the first temperature until the viable cell density reaches about 2.5 to about 3.2×10⁶ cells/mL before shifting to a different temperature. In yet another embodiment, the cell culture is grown under the first temperature until the viable cell density reaches about 2.5 to about 2.8×10⁶ cells/mL before shifting to a different temperature.

In some embodiments, the method of the present disclosure provides the temperature shift occurs 50 to 150 hours, or 60 to 140 hours, or 70 to 130 hours, or 80 to 120 hours, or 90 to 110 hours after inoculation. In some embodiments, the method of the present disclosure provides the temperature decreased about 80 hours to 150 hours after inoculation, about 90 hours to 100 hours after inoculation or about 96 hours after inoculation. In some embodiments, the temperature shift occurs 80 to 120 hours after inoculation. In some embodiments, the temperature shift occurs 90 hours, 92 hours, 94 hours, 96 hours, 98 hours, 100 hours, 102 hours, 104 hours, 106 hours, 108 hours or 110 hours after inoculation. In some embodiments, the temperature after the temperature shift is maintained until the CHO cells are harvested.

pH

Alteration of the pH of the growth medium in cell culture may affect cellular proteolytic activity, secretion, and protein production levels. Most of the cell lines grow well at about pH 7-8. Although optimum pH for cell growth varies relatively little among different cell strains, some normal fibroblast cell lines perform best at a pH 7.0-7.7 and transformed cells typically perform best at a pH of 7.0-7.4 (Eagle J Cell Physiol 82:1-8, 1973). In some embodiments, the pH of the culture medium for producing asfotase alfa is about pH 6.5-7.7 (e.g., 6.50, 6.55, 6.60, 6.65, 6.70, 6.75, 6.80, 6.85, 6.90, 6.95, 7.00, 7.05, 7.10, 7.15, 7.20, 7.25, 7.30, 7.35, 7.39, 7.40, 7.45, 7.50, 7.55, 7.60, 7.65, or 7.70).

### Culture Medium

In some embodiments, batch culture is used, wherein no additional culture medium is added after inoculation. In some embodiments, fed batch is used, wherein one or more boluses of culture medium are added after inoculation. In some embodiments, two, three, four, five or six boluses of culture medium are added after inoculation.

In various embodiments, alkaline phosphatase (e.g., asfotase alfa) is produced by a process in which extra boluses of culture medium are added to the production bioreactor. For example, one, two, three, four, five, six, or more boluses of culture medium may be added. In one particular embodiment, three boluses of culture medium are added. In various embodiments, such extra boluses of culture medium may be added in various amounts. For example, such boluses of culture medium may be added in an amount of about 20%, 25%, 30%, 33%, 40%, 45%, 50%, 60%, 67%, 70%, 75%, 80%, 90%, 100%, 110%, 120%, 125%, 130%, 133%, 140%, 150%, 160%, 167%, 170%, 175%, 180%, 190%, 200%, or more, of the original volume of culture medium in the production bioreactor. In one particular embodiment, such boluses of culture medium may be added in an amount of about 33%, 67%, 100%, or 133% of the original volume. In various embodiments, such addition of extra boluses may occur at various times during the cell growth or protein production period. For example, boluses may be added at day 1, day 2, day 3, day 4, day 5, day 6, day 7, day 8, day 9, day 10, day 11, day 12, or later in the process. In one particular embodiment, such boluses of culture medium may be added in every other day (e.g., at (1) day 3, day 5, and day 7; (2) day 4, day 6, and day 8; or (3) day 5, day 7, and day 9. In practice, the frequency, amount, time point, and other parameters of bolus supplements of culture medium may be combined freely according to the above limitation and determined by experimental practice.

Various culture mediums are available commercially. In some embodiments, the culture medium is selected from the group consisting of EX-CELL® 302 Serum-Free Medium; CD DG44 Medium; BD SELECT™ Medium; SFM4CHO Medium, or a combination thereof. In some embodiments, the culture medium comprises a combination of commercially available mediums, e.g., SFM4CHO Medium and BD SELECT™ Medium. In some embodiments, the culture medium comprises a combination of commercially available mediums, e.g., SFM4CHO Medium and BD SELECT™ Medium, at a ratio selected from 90/10, 80/20, 75/25, 70/30, 60/40, or 50/50.

### Nutrient Supplement

Various nutrient supplements, also referred to as “feed media,” are commercially available and are known to those of skill in the art. Nutrient supplements include a media (distinct from the culture media) added to a cell culture after inoculation has occurred. In some instances, the nutrient supplement can be used to replace nutrients consumed by the growing cells in the culture. In some embodiments, the nutrient supplement is added to optimize production of a desired protein, or to optimize activity of a desired protein. Numerous nutrient supplements have been developed and are available commercially. While the expressed purpose of the nutrient supplements is to increase an aspect of process development, no universal nutrient supplement exists that works for all cells and/or all proteins produced. The selection of a scalable and appropriate cell culture nutrient supplement that can work in combination with the desired cell line, protein produced and a given base medium to achieve the desired titer and growth characteristics is not routine. The typical approach of screening multiple commercially available nutrient supplements and identifying the most appropriate supplement with a specific cell line, specific protein produced and base medium combination may not be successful due to the myriad of variables present in the cell culture process. In some embodiments, the nutrient supplement is selected from the group consisting of Efficient Feed C+ AGT™ Supplement (Thermo Fisher Scientific, Waltham, MA), a combination of CELL BOOST™ 2+CELL BOOST™ 4 (GE Healthcare, Sweden), a combination of CELL BOOST™ 2+CELL BOOST™ 5 (GE Healthcare, Sweden), CELL BOOST™ 6 (GE Healthcare, Sweden), and CELL BOOST™ 7a+CELL BOOST™ 7b (GE Healthcare, Sweden), CHO feed bioreactor supplement (Sigma-Aldrich; e.g., product catalog no. C1615), or combinations thereof.

CELL BOOST™ 7a can be described as a first animal-derived component-free (ADCF) nutrient supplement comprising one or more amino acids, vitamins, salts, trace elements, poloxamer and glucose, wherein the first ADCF nutrient supplement does not comprise hypoxanthine, thymidine, insulin, L-glutamine, growth factors, peptides, proteins, hydrolysates, phenol red and 2-mercaptoethanol. CELL BOOST™ 7a is a chemically defined supplement. The phrase “animal-derived component-free” or “ADCF” refers to a supplement in which no ingredients are derived directly from an animal source, e.g., are not derived from a bovine source. In some embodiments, the nutrient supplement is CELL BOOST™ 7a.

CELL BOOST™ 7b can be described as a second ADCF nutrient supplement comprising one or more amino acids, wherein the second ADCF nutrient supplement lacks hypoxanthine, thymidine, insulin, L-glutamine, growth factors, peptides, proteins, hydrolysates, phenol red, 2-mercaptoethanol and poloxamer. CELL BOOST™ 7b is a chemically defined supplement. In some embodiments, the nutrient supplement is CELL BOOST™ 7b.

In some embodiments, combinations of commercially available nutrient supplements are used. The term “nutrient supplement” refers to both a single nutrient supplement, as well as combinations of nutrient supplements. For example, in some embodiments a combination of nutrient supplements includes a combination of CELL BOOST™ 7a and CELL BOOST™ 7b.

In various embodiments, alkaline phosphatase (e.g., asfotase alfa) is produced by a process in which extra additions of nutrient supplement are added to the production bioreactor. In some embodiments, the nutrient supplement is added over a period of time, e.g., over a period of time ranging from 1 minute to 2 hours. In some embodiments, the nutrient supplement is added in a bolus. For example, one, two, three, four, five, six, or more boluses of nutrient supplement may be added. In some embodiments, the nutrient supplement is added at more than 2 different times, e.g., 2 to 6 different times. In various embodiments, such extra boluses of nutrient supplement may be added in various amounts. For example, such boluses of nutrient supplement may be added in an amount of about 1% to 20%, 1% to 10% or 1% to 5% (w/v) of the original volume of culture medium in the production bioreactor. In one particular embodiment, such boluses of nutrient supplement may be added in an amount of 1% to 20%, 1% to 10% or 1% to 5% (w/v) of the original volume.

In some embodiments, a combination of nutrient supplements is used, and the first nutrient supplement, e.g., CELL BOOST™ 7a, is added at a concentration of 0.5% to 4% (w/v) of the culture medium. In some embodiments, a combination of nutrient supplements is used, and the second nutrient supplement, e.g., CELL BOOST™ 7b, is added at a concentration of 0.05% to 0.8% (w/v) of the culture medium. In specific embodiments wherein a combination of nutrient supplements include CELL BOOST™ 7a and CELL BOOST™ 7b, a boluses of nutrient supplement may be added in an amount of 1% to 20%, 1% to 10% or 1% to 5% (w/v) of the original volume.

In various embodiments, such addition of extra boluses may occur at various times after inoculation. For example, boluses may be added at day 1, day 2, day 3, day 4, day 5, day 6, day 7, day 8, day 9, day 10, day 11, day 12, or later after inoculation. In practice, the frequency, amount, time point, and other parameters of bolus supplements of nutrient supplement may be combined freely according to the above limitation and determined by experimental practice.

In some embodiments, the method disclosed herein further comprises adding zinc into said culture medium during production of the recombinant polypeptide. In some embodiments, zinc may be added to provide a zinc concentration of from about 1 to about 300 μM in said culture medium. In one embodiment, zinc may be added to provide a zinc concentration of from about 10 to about 200 μM (e.g., 10, 20, 30, 40, 50, 60, 70, 80, 90, 100, 110, 120, 130, 140, or 150 μM) in the culture medium. In some embodiments, zinc is added to provide a zinc concentration in the culture medium of from about 25 μM to about 150 μM, or about 60 μM to about 150 μM. In one embodiment, zinc is added to provide a zinc concentration in the culture medium of from about 30, 60, or 90 μM of zinc. In some embodiments, the zinc is added into said culture medium in a bolus, continuously, semi-continuously, or combinations thereof. In some embodiments, zinc is added one day, two days, three days, four days, five days, six days, seven days, eight days, nine days, ten days, eleven days, twelve days, and/or thirteen days after inoculation.

### Harvest

Prior studies suggested that delaying harvest timing was associated with a viability and TSAC decline, so harvest timing can have a potential impact on other CQAs. In various embodiments, alkaline phosphatase (e.g., asfotase alfa) is harvested at a time point of about 200 hr, 210 hr, 220 hr, 230 hr, 240 hr, 250 hr, 260 hr, 264 hr, 270 hr, 280 hr, 288 hr (e.g., 12 days), or more than 12 days.

### Downstream Processes

The term “downstream process(es)” used herein is generally referred to the whole or part(s) of the processes for recovery and purification of the alkaline phosphatases (e.g., asfotase alfa) produced from sources such as culture cells or fermentation broth.

Generally, downstream processing brings a product from its natural state as a component of a tissue, cell or fermentation broth through progressive improvements in purity and concentration. For example, the removal of insoluble material may be the first step, which involves the capture of the product as a solute in a particulate-free liquid (e.g., separating cells, cell debris or other particulate matter from fermentation broth). Exemplary operations to achieve this include, e.g., filtration, centrifugation, sedimentation, precipitation, flocculation, electro-precipitation, gravity settling, etc. Additional operations may include, e.g., grinding, homogenization, or leaching, for recovering products from solid sources, such as plant and animal tissues. The second step may be a “product-isolation” step, which removes components whose properties vary markedly from that of the desired product. For most products, water is the chief impurity and isolation steps are designed to remove most of it, reducing the volume of material to be handled and concentrating the product. Solvent extraction, adsorption, ultrafiltration, and precipitation may be used alone or in combinations for this step. The next step involves product purification, which separates contaminants that resemble the product very closely in physical and chemical properties. Possible purification methods include, e.g., affinity, ion-exchange chromatography, hydrophobic interaction chromatography, mixed-mode chromatography, size exclusion, reversed phase chromatography, ultrafiltration-diafiltration, crystallization and fractional precipitation. In some embodiments, the downstream processes comprise at least one of harvest clarification, ultrafiltration, diafiltration, viral inactivation, affinity capture, and combinations thereof. Downstream processes are described herein.

### Determination of Total Sialic Acid Content

In some embodiments, the methods described herein further include measuring the total sialic acid content (TSAC) of the recombinant alkaline phosphatase from an aliquot that is removed from the culture medium, e.g., from about day 6 to about day 10, particularly from about day 6 to about day 8 (e.g., about day 6, about day 7, about day 8, about day 9, about day 10, e.g., about day 7). The aliquot may be obtained aseptically from the bioreactor in order to prevent contamination. The aliquot may be from about 1 mL to about 1000 mL (e.g., from about 25 mL to about 500 mL, e.g., from about 50 mL to about 300 mL, e.g., about 100 mL or about 200 mL). Obtaining the aliquot may further include centrifuging the aliquot and/or removing the supernatant from the aliquot. This step may also include purifying the alkaline phosphatase from the supernatant using a chromatography column (e.g., a Protein A column, 1 cm Protein A column, e.g., a 1 mL HiTrap Protein A column or 600 μL Protein A Robocolumn). In some embodiments, the alkaline phosphatase may be subject to a buffer exchange. The alkaline phosphatase may also be concentrated, e.g., before determining TSAC concentration.

Commercial methods of carbohydrate quantification are available, e.g., from ThermoFisher. Generally, TSAC is released from a glycoprotein, e.g., asfotase alfa, using acid hydrolysis, and released sugars/TSAC are detected via electrochemical detection using column chromatography such as High-Performance Anion-Exchange Chromatography with Pulsed Amperometric Detection technique (HPAE-PAD). The resulting levels are quantified per mole against an internal standard and expressed as a function of the total mole protein.

As described herein, TSAC impacts the half-life of the recombinant alkaline phosphatase in physiological conditions, and thus serves as a critical quality attribute for recombinantly-produced alkaline phosphatases such as, e.g., asfotase alfa. Tight control of the TSAC range is important for reproducibility and cGMP. In some embodiments, the TSAC is about 0.8 mol/mol to about 4.0 mol/mol recombinant alkaline phosphatase. In some embodiments, the TSAC is about 0.9 mol/mol to about 3.0 mol/mol recombinant alkaline phosphatase. In some embodiments, the TSAC is about 1.0 mol/mol to about 2.8 mol/mol recombinant alkaline phosphatase. In some embodiments, the TSAC is about 1.2 mol/mol to about 3.0 mol/mol recombinant alkaline phosphatase. In some embodiments, the TSAC is about 1.2 mol/mol to about 2.4 mol/mol recombinant alkaline phosphatase. In some embodiments, the TSAC is about 0.9 mol/mol, about 1.0 mol/mol, about 1.1 mol/mol, about 1.2 mol/mol, about 1.3 mol/mol, about 1.4 mol/mol, about 1.5 mol/mol, about 1.6 mol/mol, about 1.7 mol/mol, about 1.8 mol/mol, about 1.9 mol/mol, about 2.0 mol/mol, about 2.1 mol/mol, about 2.2 mol/mol, about 2.3 mol/mol, about 2.4 mol/mol, about 2.5 mol/mol, about 2.6 mol/mol, about 2.7 mol/mol, about 2.8 mol/mol, about 2.9 mol/mol, or about 3.0 mol/mol recombinant alkaline phosphatase.

In some embodiments, the TSAC of recombinant alkaline phosphatase decreases during downstream processing. In some embodiments, the TSAC of recombinant alkaline phosphatases decreases as a result of sialidase enzymes present in the solution containing recombinant alkaline phosphatase, e.g., the cell culture, the HCCF, and/or the UFDF filtration pool. In some embodiments, sialidases are selectively removed from the cell culture, the HCCF, and/or the UFDF filtration pool to achieve a TSAC of about 0.9 mol/mol to about 3.0 mol/mol recombinant alkaline phosphatase. Sialidases can be selectively removed by, e.g., one or a combination of sialidase-specific inhibitors, antibodies, ion exchange and/or affinity chromatography, immunoprecipitation, and the like.

In some embodiments, sialic acid moieties are added to recombinant alkaline phosphatase by sialyltransferase enzymes present in the solution containing recombinant alkaline phosphatase, e.g., the cell culture, the HCCF, and/or the UFDF filtration pool. In some embodiments, recombinant sialyltransferases are added exogenously to the cell culture, the HCCF, and/or the UFDF filtration pool to achieve a TSAC of about 0.9 to about 3.0 mol/mol recombinant alkaline phosphatase.

### Determination of Recombinant Alkaline Phosphatase Activity

In some embodiments, the methods described herein further comprise measuring recombinant alkaline phosphatase activity. In some embodiments, the activity is selected from a method selected from at least one of a pNPP-based alkaline phosphatase enzymatic assay and an inorganic pyrophosphate (PPi) hydrolysis assay. In some embodiments, at least one of the recombinant alkaline phosphatase K_{cat} and Km values increases in an inorganic pyrophosphate (PPi) hydrolysis assay. In some embodiments, the method comprises determining an integral of viable cell concentration (IVCC).

The last step may be used for product polishing, the processes which culminate with packaging of the product in a form that is stable, easily transportable and convenient. Storage at 2-8° C., freezing at −20° C. to −80° C., crystallization, desiccation, lyophilization, freeze-drying and spray drying are exemplary methods in this final step. Depending on the product and its intended use, product polishing may also sterilize the product and remove or deactivate trace contaminants (e.g., viruses, endotoxins, metabolic waste products, and pyrogens), which may compromise product safety.

Product recovery methods may combine two or more steps discussed herein. For example, expanded bed adsorption (EBA) accomplishes removal of insolubles and product isolation in a single step. For a review of EBA, see Kennedy, Curr Protoc Protein Sci. 2005 June; Chapter 8: Unit 8.8. In addition, affinity chromatography often isolates and purifies in a single step.

For a review of downstream processes for purifying a recombinant protein produced in culture cells, see Rea, 2008 Solutions for Purification of Fc-fusion Proteins. BioPharm Int. Supplements March 2:20-25. The downstream processes for alkaline phosphatases disclosed herein may include at least one, or any combination, of exemplary step described herein.

### Harvest Clarification Process

In some embodiments of the method, the recombinant alkaline phosphatase is isolated from the cell culture by at least one purification step to form harvest clarified culture fluid (HCCF), e.g., a “harvesting” step or harvest clarification step. “Harvesting” the cell culture typically refers to the process of collecting the cell culture from the culture container, e.g., a bioreactor. In some embodiments, the at least one purification step comprises at least one of filtration, centrifugation, and combinations thereof. In some embodiments, the harvest clarification step comprises centrifuging and/or filtering the harvested cell culture in order to remove cells and cellular debris (e.g., insoluble biomaterials) to recover the product, e.g., the recombinant alkaline phosphatase. In some embodiments, the cells and cellular debris are removed in order to yield a clarified, filtered fluid suitable for chromatography. In some embodiments, the clarified, filtered fluid is known as harvest clarified culture fluid, or HCCF. In some embodiments, the cell culture is subjected to a combination of centrifugation and depth filtration to generate the HCCF. Possible used solutions in this step may include a recovery buffer (e.g., 50 mM Sodium Phosphate, 100 mM NaCl, pH 7.50). The composition of suitable recovery buffers may be selected by the skilled artisan.

In some embodiments, the HCCF has a total sialic acid content (TSAC) of from about 1.9 mol/mol to about 4.3 mol/mol. In some embodiments, the HCCF has a TSAC of from about 2.2 mol/mol to about 3.6 mol/mol. In some embodiments, the HCCF has a TSAC of from about 2.2 mol/mol to about 3.4 mol/mol. In some embodiments, the HCCF has a TSAC of from about 1.9 mol/mol to about 3.1 mol/mol. In some embodiments, the HCCF has a TSAC of about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5 mol/mol.

Post-Harvest Ultrafiltration and/or Diafiltration

In some embodiments of the method, an additional purification step is performed after the at least one purification step to form a filtration pool, also known as an “UFDF pool” or “UFDF.” In some embodiments, at least one purification step is for concentration and buffer dilution. In some embodiments, at least one purification step comprises at least one of harvest clarification, filtration, ultrafiltration, diafiltration, viral inactivation, affinity capture, and combinations thereof. In some embodiments, the at least one purification step comprises ultrafiltration (UF) and/or diafiltration (DF). Exemplary steps for the UF process include, e.g., pre-use cleaning/storage of the filter membrane, post-clean/post-storage flush, equilibration (e.g., with a buffer containing 50 mM sodium phosphate, 100 mM NaCl, pH 7.50), loading, concentration, diafiltration, dilution/flush/recovery (e.g., with a buffer containing 50 mM sodium phosphate, 100 mM NaCl, pH 7.50), and post-use flush/clean/storage of the filter membrane.

In some embodiments, after UF/DF, the UFDF is diluted to a protein concentration of about 1.7 g/L to about 5.3 g/L, then maintained at about 13° C. to about 27° C. for about zero to about 60 hours, prior to storage and/or further purification. “Holding” or “maintaining” the UFDF, as used herein, refers to the UFDF being kept at the same temperature (e.g., within ±about 1° C., or within a defined range, such as between 19° C. and 25° C.) for a target length of time, e.g., the “hold time” (within ±about 2 hours). The details of “holding” or “maintaining” a constant temperature may be dependent on the scale of manufacturing and practical considerations of manufacturing scale. In some embodiments, the UFDF is held in order to serve as a control point in the recombinant alkaline phosphatase production process. In some embodiments, the UFDF is held in order to ensure uniform product quality. In some embodiments, the UFDF is held in order to facilitate downstream processing.

In some embodiments, the TSAC of the recombinant alkaline phosphatase decreases during the UFDF hold time. In some embodiments, the TSAC decline is correlated with the protein concentration, length of time, and/or temperature during the UFDF hold time.

In some embodiments, the start of the UFDF hold time is immediately after the completion of diafiltration. In some embodiments, the start of the UFDF hold time is immediately after the end of the filtration step. In some embodiments, the start of the UFDF hold time is immediately after the end of the UF/DF. In some embodiments, the start of the UFDF hold time is immediately after the completion of a recirculation at the end of the UF/DF step. In some embodiments, the start of the UFDF hold time is immediately after the UF/DF product filtration and transfer is completed.

In some embodiments, the UFDF is diluted to achieve a desired protein concentration. In some embodiments, the UFDF has a protein concentration of about 1.0 g/L to about 6.0 g/L. In some embodiments, the UFDF has a protein concentration of about 1.7 g/L to about 5.3 g/L. In some embodiments, the UFDF has a protein concentration of about 1.8 g/L to about 5.0 g/L. In some embodiments, the UFDF has a protein concentration of about 2.0 g/L to about 5.0 g/L. In some embodiments, the UFDF has a protein concentration of about 1.8 g/L to about 4.3 g/L. In some embodiments, the UFDF has a protein concentration of about 2.3 g/L to about 4.3 g/L. In some embodiments, the UFDF has a protein concentration of about 3.0 g/L to about 4.5 g/L. In some embodiments, the UFDF has a protein concentration of about 3.3 g/L to about 4.1 g/L. In some embodiments, the UFDF has a protein concentration of about 1.0 g/L, about 1.1 g/L, about 1.2 g/L, about 1.3 g/L, about 1.4 g/L, about 1.5 g/L, about 1.6 g/L, about 1.7 g/L, about 1.8 g/L, about 1.9 g/L, 2.0 g/L, about 2.1 g/L, about 2.2 g/L, about 2.3 g/L, about 2.4 g/L, about 2.5 g/L, about 2.6 g/l, about 2.7 g/L, about 2.8 g/L, about 2.9 g/L, about 3.0 g/L, about 3.1 g/L, about 3.2 g/L, about 3.3 g/L, about 3.4 g/L, about 3.5 g/L, about 3.6 g/L, about 3.7 g/L, about 3.8 g/L, about 3.9 g/L, about 4.0 g/L, about 4.1 g/L, about 4.2 g/L, about 4.3 g/L, about 4.4 g/L, or about 4.5 g/L. In some embodiments, the UFDF has a protein concentration of about 2.3 g/L. In some embodiments, the UFDF has a protein concentration of about 3.1 g/L. In some embodiments, the UFDF has a protein concentration of about 3.7 g/L.

In some embodiments, the UFDF is held for about 1 hour to about 60 hours. For example, the UFDF may be held for about 1 hour (or less) to about 10 hours (e.g., 2 hours, 3 hours, 4 hours, 5 hours, 6 hours, 7 hours, 8 hours, 9 hours, or 10 hours). In some embodiments, the UFDF is held for about 10 hours to about 50 hours. In some embodiments, the UFDF is held for about 12 hours to about 48 hours. In some embodiments, the UFDF is held for about 14 hours to about 42 hours. In some embodiments, the UFDF is held for about 17 hours to about 34 hours. In some embodiments, the UFDF is held for about 19 hours to about 33 hours. In some embodiments, the UFDF is held for about 25 to about 38 hours. In some embodiments, the UFDF is held for about 29 to about 35 hours. In some embodiments, the UFDF is held for about 12 hours, about 13 hours, about 14 hours, about 15 hours, about 16 hours, about 17 hours, about 18 hours, about 19 hours, or about 20 hours. In some embodiments, the UFDF is held for about 29 hours, about 30 hours, about 31 hours, about 32 hours, about 33 hours, about 34 hours, or about 35 hours. In some embodiments, the UFDF is held for about 42 hours, about 43 hours, about 44 hours, about 45 hours, about 46 hours, about 47 hours, or about 48 hours. In some embodiments, the UFDF is held for about 14 to 20 hours. In some embodiments, the UFDF is held for about 28 to 34 hours. In some embodiments, the UFDF is held for about 42 to 48 hours.

As described above, the hold time during the filtration step (e.g., UFDF) is dependent on the TSAC concentration obtained during cell growth (e.g., from about day 6 to about day 10, e.g., from about day 6 to about day 8, e.g., on about day 7). For example, if the aliquot has a TSAC concentration of less than about 2.5 mol/mol, then the filtration step may be held for less than about nine hours. If the aliquot has a TSAC concentration of from about 2.5 mol/mol to about 2.7 mol/mol, then the filtration step may be held for from about 10 hours to about 14 hours. If the aliquot has a TSAC concentration of from about 2.8 mol/mol to about 3.0 mol/mol, then the filtration step may be held for from about 23 hours to about 27 hours. If the aliquot has a TSAC concentration of greater than about 3.0 mol/mol, then the filtration step may be held for from about 38 hours to about 42 hours.

In some embodiments, the TSAC concentration of the aliquot may be less than about 2.5 mol/mol and the filtration step is held for less than about nine hours. Alternatively, the TSAC concentration of the aliquot may be from about 2.5 mol/mol to about 2.7 mol/mol and the filtration step is held for from about 10 hours to about 14 hours.

In one alternative embodiment, if the aliquot has a TSAC concentration of less than or equal to about 2.3 mol/mol, then the filtration step may be held for about 18+/−4 hours. If the aliquot has a TSAC concentration of from about 2.4 mol/mol to about 3.1 mol/mol, then the filtration step may be held for about 32+/−4 hours. If the aliquot has a TSAC concentration of greater than or equal to about 3.2 mol/mol, then the filtration step may be held for about 44+/−4 hours.

In another alternative embodiment, if the aliquot has a TSAC concentration of less than about 2.4 mol/mol, then the filtration step may be held for about 17+/−3 hours. If the aliquot has a TSAC concentration of from about 2.4 mol/mol to about 3.6 mol/mol, then the filtration step may be held for about 31+/−3 hours. If the aliquot has a TSAC concentration of greater than about 3.6 mol/mol, then the filtration step may be held for about 45+/−3 hours.

In some embodiments, the UFDF is held at a temperature of about 10° C. to about 30° C. In some embodiments, the UFDF is held at a temperature of about 13° C. to about 27° C. In some embodiments, the UFDF is held at a temperature of about 14° C. to about 26° C. In some embodiments, the UFDF is held at a temperature of about 15° C. to about 26° C. In some embodiments, the UFDF is held at a temperature of about 15° C. to about 25° C. In some embodiments, the UFDF is held at a temperature of about 19° C. to about 25° C. In some embodiments, the UFDF is held at a temperature of about 22° C. In some embodiments, the UFDF is stored at the end of the hold time until further downstream processing steps are performed. In some embodiments, the UFDF is stored at −80° C. after flash freezing.

In some embodiments, the at least one additional purification step further comprises a viral inactivation step. In some embodiments, the viral inactivation step comprises a solvent/detergent viral inactivation process to chemically inactivate viral particles. Exemplary solvent/detergent may comprise 10% Polysorbate 80, 3% TNBP, 50 mM Sodium Phosphate, and 100 mM NaCl.

### Chromatography

In some embodiments of the method, the UFDF is subjected to at least one chromatography step to obtain partially purified recombinant alkaline phosphatase. In some embodiments, the UFDF is subjected to at least one chromatography step to obtain partially purified recombinant alkaline phosphatase, wherein the recombinant alkaline phosphatase has a total sialic acid content (TSAC) of about 0.9 mol/mol to about 3.0 mol/mol. In some embodiments, the at least one chromatography step is performed to further purify the product and/or separate the impurities/contaminants. In some embodiments, the at least one chromatography step is protein chromatography. In some embodiments, the protein chromatography is gel filtration chromatography, ion exchange chromatography, reversed-phase chromatography (RP), affinity chromatography, expanded bed adsorption (EBA), mixed-mode chromatography, and/or hydrophobic interaction chromatography (HIC). In some embodiments, the protein chromatography is affinity chromatography. In some embodiments, the protein chromatography is Protein A chromatography. In some embodiments, the Protein A chromatography captures the product (e.g., the alkaline phosphatase, such as asfotase alfa). For example, a process of GE Healthcare Mab Select SuRe Protein A chromatography may be used. Exemplary buffers and solutions used in Protein A chromatography include, e.g., equilibration/wash buffer (e.g., 50 mM Sodium Phosphate, 100 mM NaCl, pH 7.50), elution buffer (e.g., 50 mM Tris, pH 11.0), strip buffer (e.g., 100 mM Sodium Citrate, 300 mM NaCl, pH 3.2), flushing buffer, cleaning solution (e.g., 0.1 M NaOH), etc.

In some embodiments, the at least one chromatography step comprises an additional chromatography and/or purification step. In some embodiments, the at least one additional chromatography step comprises column chromatography. In some embodiments, the column chromatography is gel filtration chromatography, ion exchange chromatography, reversed-phase chromatography (RP), affinity chromatography, expanded bed adsorption (EBA), mixed-mode chromatography, and/or hydrophobic interaction chromatography (HIC). In some embodiments, the column chromatography comprises hydrophobic interaction chromatography (HIC). In some embodiments, the HIC uses Butyl Sepharose or CAPTO® Butyl agarose columns. Exemplary buffers and solutions used in a CAPTO® Butyl agarose HIC process include, e.g., loading dilution buffer/pre-equilibration buffer (e.g., 50 mM sodium phosphate, 1.4 M sodium sulfate, pH 7.50), equilibration buffer/wash buffer/elution buffer (e.g., all containing sodium phosphate and sodium sulfate), strip buffer (e.g., containing sodium phosphate), etc. Exemplary buffers and solutions used in a Butyl HIC process include, e.g., loading dilution buffer/pre-equilibration buffer (e.g., 10 mM HEPES, 2.0 M ammonium sulfate, pH 7.50), equilibration buffer/wash buffer(s)/elution buffer (e.g., all containing sodium phosphate or HEPES and ammonium sulfate), and strip buffer (e.g., containing sodium phosphate).

In some embodiments, the at least one additional purification step comprises an additional diafiltration. In some embodiments, the at least one additional chromatography and/or purification step comprises hydrophobic interaction chromatography and/or at least an additional diafiltration step. In some embodiments, the additional diafiltration step is performed after a hydrophobic interaction chromatography step. In some embodiments, the additional diafiltration step is performed for product concentration and/or buffer exchange. Exemplary buffers and solutions used in this process include, e.g., equilibration buffer (e.g., 20 mM Sodium Phosphate, 100 mM NaCl, pH 6.75), diafiltration buffer (20 mM Sodium Phosphate, 100 mM NaCl, pH 6.75), etc.

In some embodiments, the at least one additional chromatography and/or purification step is performed to obtain recombinant alkaline phosphatase with a TSAC of about 0.5 mol/mol to about 4.0 mol/mol. In some embodiments, the at least one additional chromatography and/or purification step is performed to obtain recombinant alkaline phosphatase with a TSAC of about 0.9 mol/mol to about 3.9 mol/mol. In some embodiments, the at least one additional chromatography and/or purification step is performed to obtain recombinant alkaline phosphatase with a TSAC of about 1.1 mol/mol to about 3.2 mol/mol. In some embodiments, the at least one additional chromatography and/or purification step is performed to obtain recombinant alkaline phosphatase with a TSAC of about 1.4 mol/mol to about 2.6 mol/mol. In some embodiments, the at least one additional chromatography and/or purification step is performed to obtain recombinant alkaline phosphatase with a TSAC of about 1.2 mol/mol to about 3.0 mol/mol. In some embodiments, the at least one additional chromatography step is performed to obtain recombinant alkaline phosphatase with a TSAC of about 0.8 mol/mol, about 0.9 mol/mol, 1.0 mol/mol, about 1.1 mol/mol, about 1.2 mol/mol, about 1.3 mol/mol, about 1.4 mol/mol, about 1.5 mol/mol, about 1.6 mol/mol, about 1.7 mol/mol, about 1.8 mol/mol, about 1.9 mol/mol, about 2.0 mol/mol, about 2.1 mol/mol, about 2.2 mol/mol, about 2.3 mol/mol, about 2.4 mol/mol, about 2.5 mol/mol, about 2.6 mol/mol, about 2.7 mol/mol, about 2.8 mol/mol, about 2.9 mol/mol, about 3.0 mol/mol, about 3.1 mol/mol, about 3.2 mol/mol, about 3.3 mol/mol, about 3.4 mol/mol, about 3.5 mol/mol, about 3.6 mol/mol, about 3.7 mol/mol, about 3.8 mol/mol, about 3.9 mol/mol, or about 4.0 mol/mol.

### Additional Downstream Processes

In some embodiments, additional downstream processes are performed in addition to the at least one purification step, the additional purification step, the at least one chromatography step, and/or the additional chromatography step. In some embodiments, the additional downstream processes further purify the product, e.g., the recombinant alkaline phosphatase.

In some embodiments, the additional downstream processes include a viral reduction filtration process to further remove any viral particles. In some embodiments, the viral reduction filtration process is nanofiltration.

In some embodiments, the additional downstream processes include at least one further chromatography step. In some embodiments, the at least one further chromatography step is protein chromatography. In some embodiments, the protein chromatography is gel filtration chromatography, ion exchange chromatography, reversed-phase chromatography (RP), affinity chromatography, expanded bed adsorption (EBA), mixed-mode chromatography, and/or hydrophobic interaction chromatography (HIC). In some embodiments, the third chromatography step is mixed-mode chromatography, such as CAPTO® Adhere agarose chromatography. Commercially available mixed-mode materials include, e.g., resins containing hydrocarbyl amine ligands (e.g., PPA Hypercel and HEA Hypercel from Pall Corporation, Port Washington, NY), which allow binding at neutral or slightly basic pH, by a combination of hydrophobic and electrostatic forces, and elution by electrostatic charge repulsion at low pH (see Brenac et al., 2008 J Chromatogr A. 1177:226-233); resins containing 4-mercapto-ethyl-pyridine ligand (MEP Hypercel, Pall Corporation), which achieves hydrophobic interaction by an aromatic residue and the sulfur atom facilitates binding of the target protein by thiophilic interaction (Lees et al., 2009 Bioprocess Int. 7:42-48); resins such as CAPTO® MMC mixed-mode chromatography and CAPTO® adhere agarose chromatography (GE Healthcare, Amersham, UK) containing ligands with hydrogen bonding groups and aromatic residues in the proximity of ionic groups, which leads to the salt-tolerant adsorption of proteins at different conductivities (Chen et al., 2010 J Chromatogr A. 1217:216-224); and other known chromatography materials, such as affinity resins with dye ligands, hydroxyapatite, and some ion-exchange resins (including, but not limited to, Amberlite CG 50 (Rohm & Haas, Philadelphia, PA) or Lewatit CNP 10⁵ (Lanxess, Cologne, DE). For an exemplary agarose HIC chromatography step, exemplary buffers and solutions used in this process include, e.g., pre-equilibration buffer (e.g., 0.5 M Sodium Phosphate, pH 6.00), equilibration/wash buffer (e.g., 20 mM Sodium Phosphate, 440 mM NaCl, pH 6.50), load titration buffer (e.g., 20 mM Sodium Phosphate, 3.2 M NaCl, pH 5.75), pool dilution buffer (e.g., 25 mM Sodium Phosphate, 150 mM NaCl, pH 7.40), and strip buffer (0.1 M Sodium Citrate, pH 3.20.

In some embodiments, the additional downstream processes comprise a virus filtration step for viral clearance. In some embodiments, the viral filtration step is performed by size exclusion chromatography. Exemplary buffers and solutions used in this process include, e.g., pre-use and post-product flush buffer (e.g., 20 mM Sodium Phosphate, 100 mM NaCl, pH 6.75).

In some embodiments, the additional downstream processes comprise a formulation process. In some embodiments, the formulation process comprises at least one further ultrafiltration and/or diafiltration for further concentration and/or buffer exchange. Exemplary buffers and solutions used in this process include, e.g., filter flush/equilibration/diafiltration/recovery buffer (e.g., 25 mM Sodium Phosphate, 150 mM NaCl, pH 7.40).

In some embodiments, the additional downstream processes comprise a bulk fill process. In some embodiments, the bulk fill process comprises sterile filtration. Exemplary filters for sterile filtration are Millipak 60 or Equivalent sized PVDF filters (EMD Millipore, Billerica, MA).

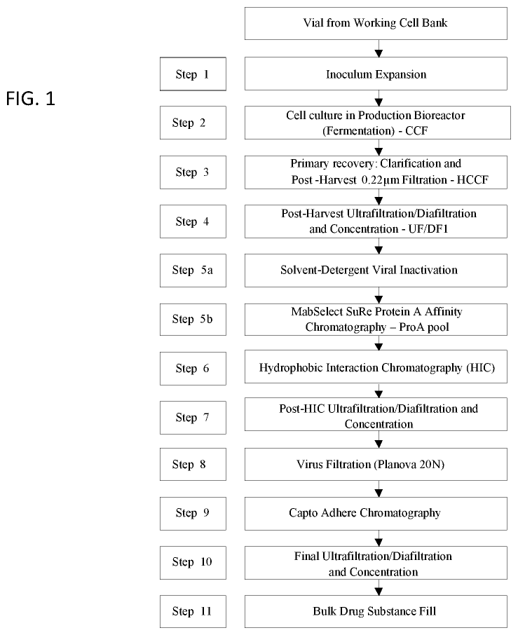

In some embodiments, the steps used for producing, purifying, and/or separating the alkaline phosphatase from the culture cells, as disclosed herein, further comprise at least one of steps selected from the group consisting of: a harvest clarification process (or a similar process to remove the intact cells and cell debris from the cell culture), an ultrafiltration (UF) process (or a similar process to concentrate the produced alkaline phosphatase), a diafiltration (DF) process (or a similar process to change or dilute the buffer comprising the produced alkaline phosphatase from previous processes), a viral inactivation process (or a similar process to inactivate or remove viral particles), an affinity capture process (or any one of chromatography methods to capture the produced alkaline phosphatase and separate it from the rest of the buffer/solution components), a formulation process and a bulk fill process. In one embodiment, the steps for producing, purifying, and/or separating the alkaline phosphatase from the culture cells, as disclosed herein, comprise at least a harvest clarification process (or a similar process to remove the intact cells and cell debris from the cell culture), a post-harvest ultrafiltration (UF) process (or a similar process to concentrate the produced alkaline phosphatase), a post-harvest diafiltration (DF) process (or a similar process to change or dilute the buffer comprising the produced alkaline phosphatase from previous processes), a solvent/detergent viral inactivation process (or a similar process to chemically inactivate viral particles), an intermediate purification process (such as hydrophobic interaction chromatography (HIC) or any one of chromatography methods to capture the produced alkaline phosphatase and separate it from the rest of the buffer/solution components), a post-HIC UF/DF process (or a similar process to concentrate and/or buffer exchange for the produced alkaline phosphatase), a viral reduction filtration process (or a similar process to further remove any viral particles or other impurities or contaminants); a mixed-mode chromatography (such as CAPTO® Adhere agarose chromatography, or a similar process to further purify and/or concentrate the produced alkaline phosphatase), a formulation process and a bulk fill process. In one embodiment, the separating step of the method provided herein further comprises at least one of harvest clarification, ultrafiltration, diafiltration, viral inactivation, affinity capture, HIC chromatography, mixed-mode chromatography and combinations thereof. FIG. 1 is an exemplary illustration of an embodiment of the production process of a recombinant alkaline phosphatase, asfotase alfa.

In some embodiments, the disclosure provides a method for controlling total sialic acid content (TSAC) in a TSAC-containing recombinant protein through mammalian cell culture, comprising at least one purification step and at least one chromatography step. In some embodiments, the disclosure provides a method for controlling glycosidase activity in mammalian cell culture producing recombinant protein, comprising at least one purification step and at least one chromatography step. In some embodiments, the at least one purification step comprises at least one of filtration, centrifugation, harvest clarification, filtration, ultrafiltration, diafiltration, viral inactivation, affinity capture, and combinations thereof. In some embodiments, the at least one chromatography step comprises protein chromatography. In some embodiments, the protein chromatography is gel filtration chromatography, ion exchange chromatography, reversed-phase chromatography (RP), affinity chromatography, expanded bed adsorption (EBA), mixed-mode chromatography, and/or hydrophobic interaction chromatography (HIC). In some embodiments, the purification step and chromatography step are ultrafiltration/diafiltration and protein A chromatography.

Following manufacture and purification, the process may produce a bulk drug substance (BDS) with a controlled range of sialyation. The methods may produce a BDS in which the TSAC concentration is controlled to a range of from about 1.2 mol/mol to about 3.0 mol/mol (e.g., from about 1.6 mol/mol to about 2.4 mol/mol). For example, the BDS may have a TSAC concentration of about 1.0 mol/mol, about 1.1 mol/mol, about 1.2 mol/mol, about 1.3 mol/mol, about 1.4 mol/mol, about 1.5 mol/mol, about 1.6 mol/mol, about 1.7 mol/mol, about 1.8 mol/mol, about 1.9 mol/mol, about 2.0 mol/mol, about 2.1 mol/mol, about 2.2 mol/mol, about 2.3 mol/mol, about 2.4 mol/mol, about 2.5 mol/mol, about 2.6 mol/mol, about 2.7 mol/mol, about 2.8 mol/mol, about 2.9 mol/mol, or about 3.0 mol/mol.

In some embodiments, the BDS is lyophilized and/or placed into vials, e.g., for distribution.

### Alkaline Phosphatases (ALPs)

The present disclosure relates to the manufacturing of an alkaline phosphatase protein (e.g., asfotase alfa) in recombinant cell culture. The alkaline phosphatase protein includes any polypeptides or molecules comprising polypeptides that comprise at least some alkaline phosphatase activity. In various embodiments, the alkaline phosphatase disclosed herein includes any polypeptide having alkaline phosphatase functions, which may include any functions of alkaline phosphatase known in the art, such as enzymatic activity toward natural substrates including phosphoethanolamine (PEA), inorganic pyrophosphate (PPi) and pyridoxal 5′-phosphate (PLP).

In certain embodiments, such alkaline phosphatase protein, after being produced and then purified by the methods disclosed herein, can be used to treat or prevent alkaline phosphatase-related diseases or disorders. For example, such alkaline phosphatase protein may be administered to a subject having decreased and/or malfunctioned endogenous alkaline phosphatase, or having overexpressed (e.g., above normal level) alkaline phosphatase substrates. In some embodiments, the alkaline phosphatase protein in this disclosure is a recombinant protein. In some embodiments, the alkaline phosphatase protein is a fusion protein. In some embodiments, the alkaline phosphatase protein in this disclosure specifically targets a cell type, tissue (e.g., connective, muscle, nervous, or epithelial tissues), or organ (e.g., liver, heart, kidney, muscles, bones, cartilage, ligaments, tendons, etc.). For example, such alkaline phosphatase protein may comprise a full-length alkaline phosphatase (ALP) or fragment of at least one alkaline phosphatase (ALP). In some embodiments, the alkaline phosphatase protein comprises a soluble ALP (sALP) linked to a bone-targeting moiety (e.g., a negatively-charged peptide as described below). In some embodiments, the alkaline phosphatase protein comprises a soluble ALP (sALP) linked to an immunoglobulin moiety (full-length or fragment). For example, such immunoglobulin moiety may comprise a fragment crystallizable region (Fc). In some embodiments, the alkaline phosphatase protein comprises a soluble ALP (sALP) linked to both a bone-targeting moiety and an immunoglobulin moiety (full-length or fragment). For more detailed description of the alkaline phosphatase protein disclosed herein, see PCT Publication Nos. WO 2005/103263 and WO 2008/138131, the teachings of both of which are incorporated by reference herein in their entirety.

In some embodiments, the alkaline phosphatase protein described herein comprises any one of the structures selected from the group consisting of: sALP-X, X-sALP, sALP-Y, Y-sALP, sALP-X-Y, sALP-Y-X, X-sALP-Y, X-Y-sALP, Y-sALP-X, and Y-X-sALP, wherein X comprises a bone-targeting moiety, as described herein, and Y comprises an immunoglobulin moiety, as described herein. In one embodiment, the alkaline phosphatase protein comprises the structure of W-sALP-X-Fc-Y-Dn/En-Z, wherein W is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; Fc is a fragment crystallizable region; Dn/En is a polyaspartate, polyglutamate, or combination thereof wherein n=8-20; and sALP is a soluble alkaline phosphatase (ALP). In some embodiments, Dn/En is a polyaspartate sequence. For example, Dn may be a polyaspartate sequence wherein n is any number between 8 and 20 (both included) (e.g., n may be 8, 9, 10, 11, 12, 13, 14, 15, 16, 17, 18, 19, and 20) (SEQ ID NO: 3). In one embodiment, Dn is D10 (SEQ ID NO: 2) or D16 (SEQ ID NO: 4). In some embodiments, Dn/En is a polyglutamate sequence. For example, En may be a polyglutamate sequence wherein n is any number between 8 and 20 (both included) (e.g., n may be 8, 9, 10, 11, 12, 13, 14, 15, 16, 17, 18, 19, or 20) (SEQ ID NO: 5). In one embodiment, En is E10 (SEQ ID NO: 6) or E16 (SEQ ID NO: 7).

For example, such sALPs may be fused to the full-length or fragment (e.g., the fragment crystallizable region (Fc)) of an immunoglobulin molecule. In some embodiments, the recombinant polypeptide comprises a structure of W-sALP-X-Fc-Y-Dn-Z, wherein W is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; Fc is a fragment crystallizable region; Dn is a poly-aspartate, poly-glutamate, or combination thereof, wherein n=10 or 16; and said sALP is a soluble alkaline phosphatase. In one embodiment, n=10. In another embodiment, W and Z are absent from said polypeptide. In some embodiments, said Fc comprises a CH2 domain, a CH3 domain and a hinge region. In some embodiments, said Fc is a constant domain of an immunoglobulin selected from the group consisting of IgG-1, IgG-2, IgG-3, IgG-3 and IgG-4. In one embodiment, said Fc is a constant domain of an immunoglobulin IgG-1. In one particular embodiment, said Fc comprises the sequence as set forth in D488-K714 of SEQ ID NO: 1.

In some embodiments, the alkaline phosphatase disclosed herein comprises the structure of W-sALP-X-Fc-Y-Dn-Z, wherein W is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; Fc is a fragment crystallizable region; Dn is a poly-aspartate, poly-glutamate, or combination thereof, wherein n=10 or 16; and said sALP is a soluble alkaline phosphatase. Such sALP is capable of catalyzing the cleavage of at least one of phosphoethanolamine (PEA), inorganic pyrophosphate (PPi) and pyridoxal 5′-phosphate (PLP). In various embodiments, the sALP disclosed herein is capable of catalyzing the cleavage of inorganic pyrophosphate (PPi). Such sALP may comprise all amino acids of the active anchored form of alkaline phosphatase (ALP) without C-terminal glycolipid anchor (GPI). Such ALP may be at least one of tissue-non-specific alkaline phosphatase (TNALP), placental alkaline phosphatase (PALP), germ cell alkaline phosphatase (GCALP), and intestinal alkaline phosphatase (IAP), or their chimeric or fusion forms or variants disclosed herein. In one particular embodiment, the ALP comprises tissue-non-specific alkaline phosphatase (TNALP). In another embodiment, the sALP disclosed herein is encoded by a polynucleotide encoding a polypeptide comprising the sequence as set forth in L1-S485 of SEQ ID NO: 1. In yet another embodiment, the sALP disclosed herein comprises the sequence as set forth in L1-S485 of SEQ ID NO: 1.

In one embodiment, the alkaline phosphatase protein comprises the structure of TNALP-Fc-D10 (SEQ ID NO: 1). Asparagine (N) residues (e.g., N 123, 213, 254, 286, 413 & 564) correspond to potential glycosylation sites. Amino acid residues (L486-K487 & D715-1716) correspond to linkers between sALP and Fc, and Fc and D10 (SEQ ID NO: 2) domains, respectively.

In this embodiment, the polypeptide is composed of five portions. The first portion (sALP) containing amino acids L1-S485 is the soluble part of the human tissue non-specific alkaline phosphatase enzyme, which contains the catalytic function. The second portion contains amino acids L486-K487 as a linker. The third portion (Fc) containing amino acids D488-K714 is the Fc part of the human immunoglobulin gamma 1 (IgG1) containing hinge, CH2 and CH3 domains. The fourth portion contains D715-1716 as a linker. The fifth portion contains amino acids D717-D726 (D10 (SEQ ID NO: 2)), which is a bone targeting moiety that allows asfotase alfa to bind to the mineral phase of bone. In addition, each polypeptide chain contains six potential glycosylation sites and eleven cysteine (Cys) residues. Cys102 exists as free cysteine. Each polypeptide chain contains four intra-chain disulfide bonds between Cys122 and Cys184, Cys472 and Cys480, Cys528 and Cys588, and Cys634 and Cys692. The two polypeptide chains are connected by two inter-chain disulfide bonds between Cys493 on both chains and between Cys496 on both chains. In addition to these covalent structural features, mammalian alkaline phosphatases are thought to have four metal-binding sites on each polypeptide chain, including two sites for zinc, one site for magnesium and one site for calcium.

There are four known isozymes of ALP, namely tissue non-specific alkaline phosphatase (TNALP) further described below, placental alkaline phosphatase (PALP) (as described e.g., in GenBank Accession Nos. NP_112603 and NP_001623), germ cell alkaline phosphatase (GCALP) (as described, e.g., in GenBank Accession No. P10696) and intestinal alkaline phosphatase (IAP) (as described, e.g., in GenBank Accession No. NP_001622). These enzymes possess very similar three-dimensional structures. Each of their catalytic sites contains four metal-binding domains, for metal ions that are necessary for enzymatic activity, including two Zn and one Mg. These enzymes catalyze the hydrolysis of monoesters of phosphoric acid and also catalyze a transphosphorylation reaction in the presence of high concentrations of phosphate acceptors. Three known natural substrates for ALP (e.g., TNALP) include phosphoethanolamine (PEA), inorganic pyrophosphate (PPi) and pyridoxal 5′-phosphate (PLP) (Whyte et al., J Clin Invest 95:1440-1445, 1995). An alignment between these isozymes is shown in FIG. 30 of WO 2008/138131, the teachings of which are incorporated by reference herein in their entirety.

The alkaline phosphatase protein in this disclosure may comprise a dimer or multimers of any ALP protein, alone or in combination. Chimeric ALP proteins or fusion proteins may also be produced, such as the chimeric ALP protein that is described in Kiffer-Moreira et al. PLoS One 9:e89374, 2014, the entire disclosure of which is incorporated by reference herein in its entirety.

In one particular embodiment, the alkaline phosphatase disclosed herein is encoded by a polynucleotide encoding a polypeptide comprising the sequence as set forth in SEQ ID NO: 1. In some embodiments, the alkaline phosphatase disclosed herein is encoded by a polynucleotide encoding a polypeptide comprising a sequence having 80%, 85%, 88%, 90%, 91%, 92%, 93%, 94%, 95%, 96%, 97%, 98% or 99% identity to SEQ ID NO: 1. In some embodiments, the alkaline phosphatase disclosed herein is encoded by a polynucleotide encoding a polypeptide comprising a sequence having 95% or 99% identity to SEQ ID NO: 1. In another embodiment, the alkaline phosphatase disclosed herein comprises the sequence as set forth in SEQ ID NO: 1.

### TNALP

As indicated above, TNALP is a membrane-bound protein anchored through a glycolipid to its C-terminus (for human TNALP, see UniProtKB/Swiss-Prot Accession No. P05186). This glycolipid anchor (GPI) is added post translationally after removal of a hydrophobic C-terminal end which serves both as a temporary membrane anchor and as a signal for the addition of the GPI. Hence, in one embodiment a soluble human TNALP comprises a TNALP wherein the first amino acid of the hydrophobic C-terminal sequence, namely alanine, is replaced by a stop codon. The soluble TNALP (herein called sTNALP) so formed contains all amino acids of the native anchored form of TNALP that are necessary for the formation of the catalytic site but lacks the GPI membrane anchor. Known TNALPs include, e.g., human TNALP [GenBank Accession Nos. NP-000469, AAI10910, AAH90861, AAH66116, AAH21289, and AAI26166]; rhesus TNALP [GenBank Accession No. XP-001109717]; rat TNALP [GenBank Accession No. NP_037191]; dog TNALP [GenBank Accession No. AAF64516]; pig TNALP [GenBank Accession No. AAN64273], mouse TNALP [GenBank Accession No. NP_031457], bovine TNALP [GenBank Accession Nos. NP_789828, NP_776412, AAM 8209, and AAC33858], and cat TNALP [GenBank Accession No. NP_001036028].

As used herein, the terminology “extracellular domain” is meant to refer to any functional extracellular portion of the native protein (e.g., without the peptide signal). Recombinant sTNALP polypeptide retaining original amino acids 1 to 501 (18 to 501 when secreted), amino acids 1 to 502 (18 to 502 when secreted), amino acids 1 to 504 (18 to 504 when secreted), or amino acids 1 to 505 (18-505 when secreted) are enzymatically active (see Oda et al., 1999 J. Biochem 126:694-699). This indicates that amino acid residues can be removed from the C-terminal end of the native protein without affecting its enzymatic activity. Furthermore, the soluble human TNALP may comprise one or more amino acid substitutions, wherein such substitution(s) does not reduce or at least does not completely inhibit the enzymatic activity of the sTNALP. For example, certain mutations that are known to cause hypophosphatasia (HPP) are listed in PCT Publication No. WO 2008/138131 and should be avoided to maintain a functional sTNALP.

### Negatively-Charged Peptide

The alkaline phosphatase protein of the present disclosure may comprise a target moiety which may specifically target the alkaline phosphatase protein to a pre-determined cell type, tissue, or organ. In some embodiments, such pre-determined cell type, tissue, or organ is bone tissues. Such bone-targeting moiety may include any known polypeptide, polynucleotide, or small molecule compounds known in the art. For example, negatively-charged peptides may be used as a bone-targeting moiety. In some embodiments, such negatively-charged peptides may be a poly-aspartate, poly-glutamate, or combination thereof (e.g., a polypeptide comprising at least one aspartate and at least one glutamate, such as a negatively-charged peptide comprising a combination of aspartate and glutamate residues). In some embodiments, such negatively-charged peptides may be D6 (SEQ ID NO: 8), D7 (SEQ ID NO: 9), D8 (SEQ ID NO: 10), D9 (SEQ ID NO: 11), D10 (SEQ ID NO: 2), D11 (SEQ ID NO: 12), D12 (SEQ ID NO: 13), D13 (SEQ ID NO: 14), D14 (SEQ ID NO: 15), D15 (SEQ ID NO: 16), D16 (SEQ ID NO: 4), D17 (SEQ ID NO: 17), D18 (SEQ ID NO: 18), D19 (SEQ ID NO: 19), D20 (SEQ ID NO: 20), or a polyaspartate having more than 20 aspartates. In some embodiments, such negatively-charged peptides may be E6 (SEQ ID NO: 21), E7 (SEQ ID NO: 22), E8 (SEQ ID NO: 23), E9 (SEQ ID NO: 24), E10 (SEQ ID NO: 6), E11 (SEQ ID NO: 25), E12 (SEQ ID NO: 26), E13 (SEQ ID NO: 27), E14 (SEQ ID NO: 28), E15 (SEQ ID NO: 29), E16 (SEQ ID NO: 7), E17 (SEQ ID NO: 30), E18 (SEQ ID NO: 31), E19 (SEQ ID NO: 32), E20 (SEQ ID NO: 33), or a polyglutamate having more than 20 glutamates. In one embodiment, such negatively-charged peptides may comprise at least one selected from the group consisting of D10 (SEQ ID NO: 2) to D16 (SEQ ID NO: 4) or E10 (SEQ ID NO: 6) to E16 (SEQ ID NO: 7).

### Spacer

In some embodiments, the alkaline phosphatase protein of the present disclosure comprises a spacer sequence between the ALP portion and the targeting moiety portion. In one embodiment, such alkaline phosphatase protein comprises a spacer sequence between the ALP (e.g., TNALP) portion and the negatively-charged peptide targeting moiety. Such spacer may be any polypeptide, polynucleotide, or small molecule compound. In some embodiments, such spacer may comprise fragment crystallizable region (Fc) fragments. Useful Fc fragments include Fc fragments of IgG that comprise the hinge, and the CH2 and CH3 domains. Such IgG may be any of IgG-1, IgG-2, IgG-3, IgG-3 and IgG-4, or any combination thereof.

Without being limited to this theory, it is believed that the Fc fragment used in bone-targeted sALP fusion proteins (e.g., asfotase alfa) acts as a spacer, which allows the protein to be more efficiently folded given that the expression of sTNALP-Fc-D10 was higher than that of sTNALP-D10. One possible explanation is that the introduction of the Fc fragment alleviates the repulsive forces caused by the presence of the highly negatively-charged D10 sequence (SEQ ID NO: 2) added at the C-terminus of the sALP sequence exemplified herein. In some embodiments, the alkaline phosphatase protein described herein comprises a structure selected from the group consisting of: sALP-Fc-D10, sALP-D10-Fc, D10-sALP-Fc, D10-Fc-sALP, Fc-sALP-D10, and Fc-D10-sALP. In other embodiments, the D10 (SEQ ID NO: 2) in the above structures is substituted by other negatively-charged polypeptides (e.g., D8 (SEQ ID NO: 10), D16 (SEQ ID NO: 4), E10 (SEQ ID NO: 6), E8 (SEQ ID NO: 23), E16 (SEQ ID NO: 7), etc.).

Useful spacers for the present disclosure include, e.g., polypeptides comprising a Fc, and hydrophilic and flexible polypeptides able to alleviate the repulsive forces caused by the presence of the highly negatively-charged bone-targeting sequence (e.g., D10 (SEQ ID NO: 2)) added at the C-terminus of the sALP sequence.

### Dimers/Tetramers

In specific embodiments, the bone-targeted sALP fusion proteins of the present disclosure are associated so as to form dimers or tetramers.

In the dimeric configuration, the steric hindrance imposed by the formation of the interchain disulfide bonds is presumably preventing the association of sALP domains to associate into the dimeric minimal catalytically-active protein that is present in normal cells.

The bone-targeted sALP may further optionally comprise one or more additional amino acids 1) downstream from the negatively-charged peptide (e.g., the bone tag); and/or 2) between the negatively-charged peptide (e.g., the bone tag) and the Fc fragment; and/or 3) between the spacer (e.g., an Fc fragment) and the sALP fragment. This could occur, for example, when the cloning strategy used to produce the bone-targeting conjugate introduces exogenous amino acids in these locations. However the exogenous amino acids should be selected so as not to provide an additional GPI anchoring signal. The likelihood of a designed sequence being cleaved by the transamidase of the host cell can be predicted as described by Ikezawa, 2002 Glycosylphosphatidylinositol (GPI)-anchored proteins. Biol Pharm Bull. 25:409-17.

The present disclosure also encompasses a fusion protein that is post-translationally modified, such as by glycosylation including those expressly mentioned herein, acetylation, amidation, blockage, formylation, gamma-carboxyglutamic acid hydroxylation, methylation, phosphorylation, pyrrolidone carboxylic acid, and sulfation.

### Asfotase Alfa

Asfotase alfa is a soluble Fc fusion protein consisting of two TNALP-Fc-D10 polypeptides each with 726 amino acids as shown in SEQ ID NO: 1. Each polypeptide or monomer is composed of five portions. The first portion (sALP) containing amino acids L1-S485 is the soluble part of the human tissue non-specific alkaline phosphatase enzyme, which contains the catalytic function. The second portion contains amino acids L486-K487 as a linker. The third portion (Fc) containing amino acids D488-K714 is the Fc part of the human Immunoglobulin gamma 1 (IgG1) containing hinge, CH2 and CH3 domains. The fourth portion contains D715-1716 as a linker. The fifth portion contains amino acids D717-D726 (D10 (SEQ ID NO: 2)), which is a bone targeting moiety that allows asfotase alfa to bind to the mineral phase of bone. In addition, each polypeptide chain contains six potential glycosylation sites and eleven cysteine (Cys) residues. Cys102 exists as free cysteine. Each polypeptide chain contains four intra-chain disulfide bonds between Cys122 and Cys184, Cys472 and Cys480, Cys528 and Cys588, and Cys634 and Cys692. The two polypeptide chains are connected by two inter-chain disulfide bonds between Cys493 on both chains and between Cys496 on both chains. In addition to these covalent structural features, mammalian alkaline phosphatases are thought to have four metal-binding sites on each polypeptide chain, including two sites for zinc, one site for magnesium and one site for calcium.

Asfotase alfa can also be characterized as follows. From the N-terminus to the C terminus, asfotase alfa comprises: (1) the soluble catalytic domain of human tissue non-specific alkaline phosphatase (TNSALP) (UniProtKB/Swiss-Prot Accession No. P05186), (2) the human immunoglobulin G1 Fc domain (UniProtKB/Swiss-Prot Accession No. P01857) and (3) a deca-aspartate peptide (D10 (SEQ ID NO: 2)) used as a bone-targeting domain (Nishioka et al. 2006 Mol Genet Metab 88:244-255). The protein associates into a homo-dimer from two primary protein sequences. This fusion protein contains 6 confirmed complex N-glycosylation sites. Five of these N-glycosylation sites are located on the sALP domain and one on the Fc domain. Another important post-translational modification present on asfotase alfa is the presence of disulfide bridges stabilizing the enzyme and the Fc-domain structure. A total of 4 intra-molecular disulfide bridges are present per monomer and 2 inter-molecular disulfide bridges are present in the dimer. One cysteine of the alkaline phosphatase domain is free.

Asfotase alfa has been used as an enzyme-replacement therapy for the treatment of hypophosphatasia (HPP). In patients with HPP, loss-of-function mutation(s) in the gene encoding TNSALP causes a deficiency in TNSALP enzymatic activity, which leads to elevated circulating levels of substrates, such as inorganic pyrophosphate (PPi) and pyridoxal-5′-phosphate (PLP). Administration of asfotase alfa to patients with HPP cleaves PPi, releasing inorganic phosphate for combination with calcium, thereby promoting hydroxyapatite crystal formation and bone mineralization, and restoring a normal skeletal phenotype. For more details on asfotase alfa and its uses in treatment, see PCT Publication Nos. WO 2005/103263 and WO 2008/138131

In some embodiments, the method provides an alkaline phosphatase (asfotase alfa) having improved enzymatic activity of the produced alkaline phosphatase (e.g., asfotase alfa) relative to an alkaline phosphatase produced by conventional means, by minimizing the concentration of metal ions having potential negative impact on activity or increasing the concentration of metal ions having potential positive impact on activity or both as described herein. Activity may be measured by any known method. Such methods include, e.g., those in vitro and in vivo assays measuring the enzymatic activity of the produced alkaline phosphatase (e.g., asfotase alfa) to substrates of an alkaline phosphatase, such as phosphoethanolamine (PEA), inorganic pyrophosphate (PPi) and pyridoxal 5′-phosphate (PLP).

In some embodiments, the alkaline phosphatase disclosed herein is encoded by a first polynucleotide which hybridizes under high stringency conditions to a second polynucleotide comparing the sequence completely complementary to a third polynucleotide encoding a polypeptide comprising the sequence as set forth in SEQ ID NO: 1. Such high stringency conditions may comprise: pre-hybridization and hybridization in 6×SSC, 5×Denhardt's reagent, 0.5% SDS and 100 mg/ml of denatured fragmented salmon sperm DNA at 68° C.; and washes in 2×SSC and 0.5% SDS at room temperature for 10 minutes; in 2×SSC and 0.1% SDS at room temperature for 10 minutes; and in 0.1×SSC and 0.5% SDS at 65° C. three times for 5 minutes.

### EXAMPLES

### Example 1: Asfotase Alfa Manufacturing Process

An exemplary asfotase alfa bulk drug substance (BDS) manufacturing process is shown in FIG. 1.

Described in detail below is an asfotase alfa manufacturing process in which TSAC content was measured at Day 7 of the cell culture fermentation in a production bioreactor and used to determine the hold time for a downstream post-harvest ultrafiltration/diafiltration (UF/DF1) step. This added step provided increased quality control in which the final TSAC content was maintained within the acceptable range that has been previously approved for use in humans. The manufacturing process and target TSAC range provide a final drug product with proper enzymatic activity, a therapeutically effective half-life, and repeatability between batches.

### History of In-Process TSAC Controls

Sialic acid is a known form of glycosylation associated with asfotase alfa that impacts half-life of the molecule in physiological conditions. Controlling TSAC levels within the acceptable range of 1.2-3.0 mol sialic acid/mol asfotase alfa monomer (mol/mol) is necessary to provide final drug product with proper efficacy, a therapeutically effective half-life, and repeatability between batches. TSAC is generated in the production bioreactor (cell culture fluid, CCF, step 2 in FIG. 1) and TSAC in the harvested cell culture fluid (HCCF, step 3 in FIG. 1) decreases during the post-harvest ultrafiltration/diafiltration (UF/DF1) (step 4 in FIG. 1) pool hold prior to the Protein A, MabSelect™ SuRe™ (ProA), chromatography step (step 5b in FIG. 1).

The UF/DF1 pool hold is an important in-process control step for BDS TSAC. Prior small scale characterization studies established that the post-harvest UF/DF1 hold time, protein concentration and temperature significantly impact the magnitude to which TSAC decreases during the UF/DF1 pool hold.

### Manufacturing TSAC Data Review

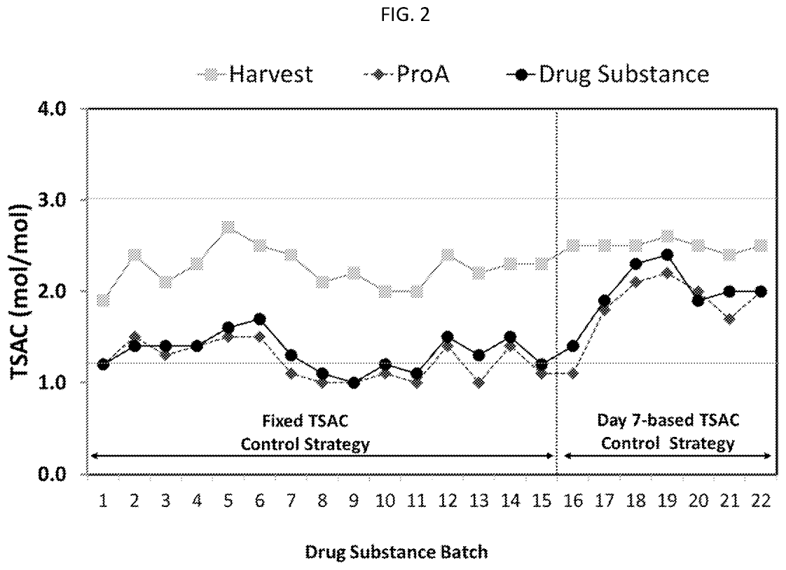

TSAC is measured at two steps during manufacturing (ProA pool and BDS release). For a subset of batches, TSAC has also been measured at the production bioreactor step (Day 7 and Day 10 referred to as CCF), harvest step (HCCF)) and the HIC step. The TSAC data for 20,000 L batches are tabulated in Table 1. A review of the manufacturing data confirmed that TSAC decreases from the HCCF step (step 3 in FIG. 1) to the ProA step (step 5b of FIG. 1) as tabulated in Table 1 and shown in FIG. 2.

The TSAC at BDS results were observed to be trending near the lower specification limit, including three results ((#8, #9, and #11) which were out of specification (OOS). Variability in the upstream process, specifically the production bioreactor, was identified as a probable cause for BDS OOS TSAC. Additionally, the TSAC controls downstream of the production bioreactor (UF/DF1 hold) were not optimally designed to respond to this upstream variability.

TABLE 1
In-Process and BDS TSAC Data and UF/DF1 Process Data

| | | | TSAC at | | | | | | | |
|---|---|---|---|---|---|---|---|---|---|---|
| | | | CCF | | | | | Average | UF/DF1 | UF/DF1 |
| | | Day 7 | (pre- | TSAC at | ProA | HIC | BDS | UF/DF1 Hold | Protein | Hold |
| TSAC | BDS | TSAC² | harvest) | HCCF | TSAC | TSAC⁴ | TSAC | Temperature | Concentration | Time |
| Control | Batch | (mol/mol) | (mol/mol) | (mol/mol) | (mol/mol) | (mol/mol) | (mol/mol) | (° C.) | (g/L) | (hr) |
| Fixed | 1 | 2.4 | 1.9 | 1.9 | 1.2 | 1.3 | 1.2 | 22.0 | 3.2 | 31 |
| TSAC | 2 | 3.0 | 2.3 | 2.4 | 1.5 | 1.6 | 1.4 | 22.0 | 3.2 | 32 |
| control | 3 | 2.1 | 2.4 | 2.1 | 1.3 | 1.6 | 1.4 | 22.0 | 3.1 | 25 |
| strategy | 4 | N/A | 2.1 | 2.3 | 1.4 | N/A | 1.4 | 22.0 | 3.5 | 20 |
| | 5 | N/A | 2.4 | 2.7 | 1.5 | N/A | 1.6 | 22.0 | 3.1 | 19 |
| | 6 | N/A | 2.3 | 2.5 | 1.5 | N/A | 1.7 | 22.0 | 3.0 | 19 |
| | 7 | N/A | 2.1 | 2.4 | 1.1 | N/A | 1.3 | 22.0 | 3.1 | 19 |
| | 8¹ | N/A | N/A³ | 2.1 | 1.0 | N/A | 1.1 | 22.0 | 2.9 | 19 |
| | 9¹ | N/A | 1.8 | 2.2 | 1.0 | N/A | 1.0 | 22.0 | 3.1 | 19 |
| | 10 | N/A | 1.8 | 2.0 | 1.1 | N/A | 1.2 | 22.0 | 3.0 | 19 |
| | 11¹ | N/A | 1.7 | 2.0 | 1.0 | N/A | 1.1 | 22.0 | 3.1 | 24 |
| | 12 | N/A | 2.1 | 2.4 | 1.4 | N/A | 1.5 | 22.0 | 3.2 | 14 |
| | 13 | N/A | 1.9 | 2.2 | 1.0 | N/A | 1.3 | 22.0 | 3.4 | 14 |
| | 14 | N/A | 1.9 | 2.3 | 1.4 | N/A | 1.5 | 22.0 | 3.0 | 14 |
| | 15 | 2.0 | 1.8 | 2.3 | 1.1 | N/A | 1.2 | 22.0 | 3.1 | 15 |
| Day 7 | 16⁵ | 2.6 | 2.1 | 2.5 | 1.1 | 1.3 | 1.4 | 22.0 | 2.3 | 18 |
| Based | 17⁶ | 2.1 | 2.0 | 2.5 | 1.8 | 1.9 | 1.9 | 22.0 | 2.5 | 6 |
| TSAC | 18 | 2.4 | 2.2 | 2.5 | 2.1 | 2.4 | 2.3 | 22.0 | 2.1 | 4 |
| control | 19 | 2.3 | 2.1 | 2.6 | 2.2 | 2.4 | 2.4 | 22.0 | 2.5 | 4 |
| strategy | 20 | 2.4 | 2.0 | 2.5 | 2.0 | N/A | 1.9 | 22.0 | 2.3 | 4 |
| | 21 | 2.4 | 2.1 | 2.4 | 1.7 | N/A | 2.0 | 22.0 | 2.5 | 3 |
| | 22 | 2.2 | 2.0 | 2.5 | 2.0 | N/A | 2.0 | 22.0 | 2.4 | 9 |

¹BDS out of specification for TSAC
²Day 7 TSAC is not routinely tested during manufacture of batches with Fixed TSAC control.
³Sample was not analyzed for TSAC due to sample mishandling prior to small scale purification and assay execution.
⁴HIC TSAC is not routinely tested during manufacture.
⁵UF/DF1 hold time was within target range defined by Day 7 action limits at the time of batch execution. Based on final enhanced TSAC control strategy, the target hold time for this batch would be 10 to 14 hours per Table 3.
⁶A fixed UF/DF1 hold time was implemented to demonstrate operational feasibility. The hold time achieved was within the target range defined by Day 7 action limits per Table 3 (< 9 hours).

### Enhanced TSAC Control Strategy

An enhanced TSAC control strategy was developed. The enhanced strategy involves monitoring the TSAC levels in the production bioreactor and modulating the UF/DF1 process parameters in response to improve process control and capability of TSAC at BDS. Specifically, TSAC measured at Day 7 from a production bioreactor sample (referred to as “Day 7 TSAC”) can provide an estimate for TSAC prior to the UF/DF1 unit operation. Day 7 TSAC has been introduced as an in-process control (IPC) for the process and the associated action limits for Day 7 TSAC identify the target UF/DF1 hold time for each batch. The Day 7 TSAC is used as a surrogate for a TSAC result from end of cell culture or post-harvest since the sample preparation (small scale Protein A purification) and TSAC assay durations currently preclude actual at-line measurement of TSAC prior to start of UF/DF1 operation and hold. In order to optimize the UF/DF1 hold strategy in response to batch-specific TSAC production in the bioreactor, the protein concentration target and range were adjusted. The UF/DF1 hold temperature was unchanged.

The changes introduced in the UF/DF1 operation to further enhance TSAC control are summarized in Table 2. Day 7 TSAC action limits are listed in Table 3. The parameter and attribute ranges and IPC action limits were optimized using the predictive model generated from small scale UF/DF1 characterization studies and additional TSAC data collection from manufacturing-scale batches.

TABLE 2
Summary of enhanced TSAC control

| | | TSAC | |
|---|---|---|---|
| Parameter/ | Original | Control | |
| Attribute | Range | Range | Rationale |
| Day 7 TSAC | N/A | Refer to | Day 7 TSAC was monitored and used to guide |
| (mol/mol) | | Table 3 | adjustment of target UF/DF1 hold time in order to |
| | | | improve process control and capability of TSAC |
| | | | at BDS |
| UF/DF1 Hold | 14-42 | 0-48 | Parameter was controlled to a target based on |
| Time (hr) | | | Day 7 TSAC action limits. Refer to Table 3. |
| | | | Wider acceptable range allowed response to |
| | | | wider range of TSAC generated in production |
| | | | bioreactor. |
| UF/DF1 Hold | 15-25 | 15-25 | No change |
| Temperature (° C.) | | | |
| UF/DF1 Protein | 2.0-4.3 | 1.8-4.3 | Wider range was optimized for changes to hold |
| Concentration | | | time strategy and enabled response to wider |
| (g/L) | | | range of TSAC generated in production |
| | | | bioreactor. |

TABLE 3
Day 7 TSAC and UF/DF1 Hold time targets for enhanced TSAC control

| | Action in response to Day 7 TSAC result: |
|---|---|
| | Target UF/DF1 Hold Time |
| Day 7 TSAC | Target Range |
| (mol/mol) | (hr) |
| <2.5 | <9 |
| 2.5-2.7 | 10-14 |
| 2.8-3.0 | 23-27 |
| >3.0 | 38-42 |

The updates to the TSAC control strategy improved process control and capability of TSAC at BDS over a wider range of TSAC generated during the cell culture process in the production bioreactor. Development batches were used to fine-tune the UF/DF1 hold times, demonstrate manufacturing operational feasibility of the Day 7 TSAC in-process control, and demonstrate the effectiveness of the modified control strategy.

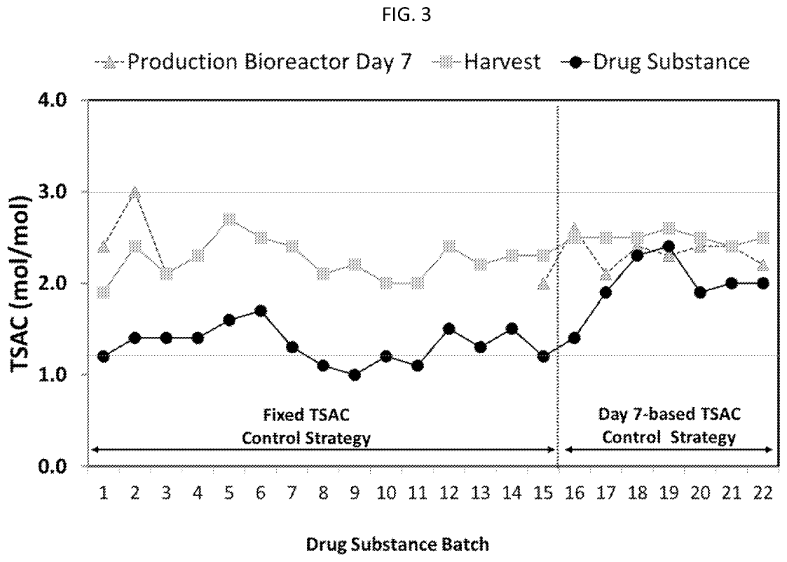

Four initial development batches were successfully executed through to BDS (batches 16, 17, 18, and 19; see Table 1 and FIGS. 2 and 3). The Day 7 samples from the production bioreactor were purified using small scale Protein A chromatography and then tested for TSAC. For three of the four development batches, the Day 7 TSAC result was used to modulate the UF/DF1 hold time based on pre-defined action limits similar to those specified in Table 3. One batch (17) was executed with a fixed hold time target in order to demonstrate operational feasibility for the shortest hold times below the previously defined range. Three additional batches were manufactured through to BDS (batches 20 to 22) with the enhanced TSAC control strategy. The TSAC and UF/DF1 process data for all batches with enhanced TSAC control are summarized in Table 1. For all batches, the UF/DF1 temperature, protein concentration, and hold time were within the acceptable ranges defined for the enhanced control strategy (Table 2). Actual UF/DF1 hold times were within the target ranges defined for Day 7 action limits (Table 3), except the first development batch 16 which was executed with different Day 7 action limits.

The TSAC from the production bioreactor (CCF) and post-harvest (HCCF) (HCCF shown in FIG. 2) trend similarly in the enhanced TSAC control batches compared to previous batches and Day 7 TSAC approximates the TSAC prior to the UF/DF1 unit operation (HCCF TSAC) (Table 1, FIG. 2). The mean BDS TSAC for batches 1 to 15 without Day 7 TSAC control (n=15) was 1.3 mol/mol, compared with a mean BDS TSAC of 2.1 mol/mol (range 1.9 to 2.4 mol/mol) for batches which utilized the Day 7 TSAC control (batches 17 to 22, n=6). The shift in BDS TSAC for batches 17 to 22, closer to the midpoint of the specification range compared to prior batches, is consistent with, and illustrates the efficacy of, the UF/DF1 hold conditions executed for these batches using the enhanced TSAC control (Table 1 and FIG. 2 and FIG. 3). While the TSAC from the production bioreactor (Day 7) in these batches has resulted in shorter target UF/DF1 hold times, the enhanced TSAC control strategy responds dynamically to a range of TSAC outputs from the production bioreactor. If a higher TSAC output is measured at Day 7, the enhanced strategy identifies and defines an appropriate target UF/DF1 hold time in order to shift TSAC at BDS closer to the midpoint of the specification range and avoid OOS results.

In addition to demonstrating feasibility and efficacy of the enhanced TSAC control strategy, the BDS from batches 17 to 22 meet all criteria per the release specification, confirming no adverse impact to process performance or to other product quality attributes.

### OTHER EMBODIMENTS

All references cited herein are incorporated by reference in their entirety. Although the foregoing disclosure has been described in some detail by way of illustration and example for purposes of clarity of understanding, it is apparent to those skilled in the art that certain minor changes and modifications will be practiced. Therefore, the description and examples should not be construed as limiting the scope of the disclosure.

The foregoing detailed description and examples have been given for clarity of understanding only. No unnecessary limitations are to be understood therefrom. The disclosure is not limited to the exact details shown and described, for variations apparent to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

The complete disclosures of all patents, patent applications including provisional patent applications, publications including patent publications and nonpatent publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The disclosure is not limited to the exact details shown and described, for variations apparent to one skilled in the art will be included within the embodiments defined by the claims.

1. A method of producing a recombinant alkaline phosphatase comprising:
(a) inoculating a bioreactor with a cell expressing a recombinant alkaline phosphatase;
(b) obtaining an aqueous culture medium comprising the recombinant alkaline phosphatase;
(c) obtaining an aliquot from the aqueous culture medium at from about day 6 to about day 10 after inoculation;
(d) quantifying the total sialic acid content (TSAC) molar concentration per mole of the recombinant alkaline phosphatase in the aliquot;
(e) harvesting the aqueous culture medium; and
(f) performing at least one purification step to obtain a bulk drug solution (BDS);
wherein:
(i)
(1) the aliquot comprises a TSAC concentration of less than about 2.5 mol/mol and the filtration step is held for less than about nine hours;
(2) the aliquot comprises a TSAC concentration of from about 2.5 mol/mol to about 2.7 mol/mol and the filtration step is held for from about 10 hours to about 14 hours;
(3) the aliquot comprises a TSAC concentration of from about 2.8 mol/mol to about 3.0 mol/mol and the filtration step is held for from about 23 hours to about 27 hours; or
(4) the aliquot comprises a TSAC concentration of greater than about 3.0 mol/mol and the filtration step is held for from about 38 hours to about 42 hours; or
(ii)
(1) the aliquot comprises a TSAC concentration of less than about 2.5 mol/mol and the filtration step is held for from about five hours to about nine hours;
(2) the aliquot comprises a TSAC concentration of from about 2.5 mol/mol to about 2.7 mol/mol and the filtration step is held for from about 16 hours to about 20 hours; or
(3) the aliquot comprises a TSAC concentration of greater than about 2.7 mol/mol and the filtration step is held for from about 30 hours to about 34 hours; or
(iii)
(1) the aliquot comprises a TSAC concentration of less than or equal to about 2.3 mol/mol and the filtration step is held for from about 14 hours to about 22 hours;
(2) the aliquot comprises a TSAC concentration of from about 2.4 mol/mol to about 3.1 mol/mol and the filtration step is held for from about 28 hours to about 36 hours; or
(3) the aliquot comprises a TSAC concentration of greater than or equal to about 3.2 mol/mol and the filtration step is held for from about 40 hours to about 48 hours.

2. The method of claim 1, wherein step (c) comprises obtaining the aliquot from the aqueous culture medium at about day 7 after inoculation.

3. The method of claim 1, wherein the filtration step comprises ultrafiltration, diafiltration, or a combination thereof.

4. (canceled)

5. The method of claim 1, wherein the cell is a Chinese Hamster Ovary (CHO) cell.

6. The method of claim 1, wherein the TSAC concentration of the aliquot is less than about 2.5 mol/mol and the filtration step is held for less than about nine hours.

7. The method of claim 1, wherein the TSAC concentration of the aliquot is from about 2.5 mol/mol to about 2.7 mol/mol and the filtration step is held for about 10 hours to about 14 hours.

8. The method of claim 1, wherein the alkaline phosphatase concentration during the filtration step is about 1.8 g/L to about 5.0 g/L; and/or the TSAC concentration of the BDS is about 1.2 mol/mol to about 3.0 mol/mol.

9. The method of claim 8, wherein the alkaline phosphatase concentration during the filtration step is about 1.8 to about 4.3 g/L; and/or the TSAC concentration of the BDS is about 1.6 mol/mol to about 2.4 mol/mol.

10-13. (canceled)

14. The method of claim 1, wherein the filtration step is held at a constant temperature is of about 15° C. to about 25° C.

15. (canceled)

16. The method of claim 14, wherein the temperature is about 22° C.

17. (canceled)

18. The method of claim 1, wherein the aliquot is from about 1 ml to about 500 ml.

19-20. (canceled)

21. The method of claim 1, wherein step (c) further comprises centrifuging the aliquot, removing a supernatant from the aliquot, and, optionally, purifying the alkaline phosphatase from the supernatant using a chromatography column.

22-23. (canceled)

24. The method of claim 21, wherein the chromatography column comprises a Protein A column.

25. (canceled)

26. The method of claim 21, wherein step (c) further comprises performing a buffer exchange and/or concentrating the alkaline phosphatase.

27. (canceled)

28. The method of claim 1, wherein step (d) comprises performing acid hydrolysis to release the TSAC.

29. The method of claim 1, further comprising lyophilizing the alkaline phosphatase.

30. (canceled)

31. The method of claim 1, wherein the bioreactor has a volume of at least 2 L, at least 10 L, at least 1,000 L, at least 10,000 L, or at least 20,000 L.

32-35. (canceled)

36. The method of claim 1, wherein the culture medium is selected from the group consisting of EX-CELL® 302 Serum-Free Medium; CD DG44 Medium; BD SELECT™ Medium; SFM4CHO Medium; and combinations thereof.

37. The method of claim 1, wherein the recombinant alkaline phosphatase comprises the structure of W-sALP-X-Fc-Y-Dn-Z, wherein:
W is absent or is an amino acid sequence of at least one amino acid;
X is absent or is an amino acid sequence of at least one amino acid;
Y is absent or is an amino acid sequence of at least one amino acid;
Z is absent or is an amino acid sequence of at least one amino acid;
Fc is a fragment crystallizable region;
Dn is a poly-aspartate, poly-glutamate, or combination thereof, wherein n=10 or 16; and
sALP is a soluble alkaline phosphatase.

38. The method of claim 37, wherein the recombinant alkaline phosphatase comprises an amino acid sequence having at least 90% or 100% sequence identity to the sequence set forth in SEQ ID NO: 1.

39. (canceled)


SEQUENCE LISTING (33 sequences)

SEQ ID NO: 1 - protein, 726 aa, Artificial Sequence
  Description of Artificial Sequence Synthetic polypeptide
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD

SEQ ID NO: 2 - protein, 10 aa, Artificial Sequence
  Description of Artificial Sequence Synthetic peptide
DDDDDDDDDD

SEQ ID NO: 3 - protein, 20 aa, Artificial Sequence
  Description of Artificial Sequence Synthetic peptide
  SITE (1)..(20): This sequence may encompass 8-20 residues
DDDDDDDDDDDDDDDDDDDD

SEQ ID NO: 4 - protein, 16 aa, Artificial Sequence
  Description of Artificial Sequence Synthetic peptide
DDDDDDDDDDDDDDDD

SEQ ID NO: 5 - protein, 20 aa, Artificial Sequence
  Description of Artificial Sequence Synthetic peptide
  SITE (1)..(20): This sequence may encompass 8-20 residues
EEEEEEEEEEEEEEEEEEEE

SEQ ID NO: 6 - protein, 10 aa, Artificial Sequence
  Description of Artificial Sequence Synthetic peptide
EEEEEEEEEE

SEQ ID NO: 7 - protein, 16 aa, Artificial Sequence
  Description of Artificial Sequence Synthetic peptide
EEEEEEEEEEEEEEEE

SEQ ID NO: 8 - protein, 6 aa, Artificial Sequence
  Description of Artificial Sequence Synthetic peptide
DDDDDD

SEQ ID NO: 9 - protein, 7 aa, Artificial Sequence
  Description of Artificial Sequence Synthetic peptide
DDDDDDD

SEQ ID NO: 10 - protein, 8 aa, Artificial Sequence
  Description of Artificial Sequence Synthetic peptide
DDDDDDDD

SEQ ID NO: 11 - protein, 9 aa, Artificial Sequence
  Description of Artificial Sequence Synthetic peptide
DDDDDDDDD

SEQ ID NO: 12 - protein, 11 aa, Artificial Sequence
  Description of Artificial Sequence Synthetic peptide
DDDDDDDDDDD

SEQ ID NO: 13 - protein, 12 aa, Artificial Sequence
  Description of Artificial Sequence Synthetic peptide
DDDDDDDDDDDD

SEQ ID NO: 14 - protein, 13 aa, Artificial Sequence
  Description of Artificial Sequence Synthetic peptide
DDDDDDDDDDDDD

SEQ ID NO: 15 - protein, 14 aa, Artificial Sequence
  Description of Artificial Sequence Synthetic peptide
DDDDDDDDDDDDDD

SEQ ID NO: 16 - protein, 15 aa, Artificial Sequence
  Description of Artificial Sequence Synthetic peptide
DDDDDDDDDDDDDDD

SEQ ID NO: 17 - protein, 17 aa, Artificial Sequence
  Description of Artificial Sequence Synthetic peptide
DDDDDDDDDDDDDDDDD

SEQ ID NO: 18 - protein, 18 aa, Artificial Sequence
  Description of Artificial Sequence Synthetic peptide
DDDDDDDDDDDDDDDDDD

SEQ ID NO: 19 - protein, 19 aa, Artificial Sequence
  Description of Artificial Sequence Synthetic peptide
DDDDDDDDDDDDDDDDDDD

SEQ ID NO: 20 - protein, 20 aa, Artificial Sequence
  Description of Artificial Sequence Synthetic peptide
DDDDDDDDDDDDDDDDDDDD

SEQ ID NO: 21 - protein, 6 aa, Artificial Sequence
  Description of Artificial Sequence Synthetic peptide
EEEEEE

SEQ ID NO: 22 - protein, 7 aa, Artificial Sequence
  Description of Artificial Sequence Synthetic peptide
EEEEEEE

SEQ ID NO: 23 - protein, 8 aa, Artificial Sequence
  Description of Artificial Sequence Synthetic peptide
EEEEEEEE

SEQ ID NO: 24 - protein, 9 aa, Artificial Sequence
  Description of Artificial Sequence Synthetic peptide
EEEEEEEEE

SEQ ID NO: 25 - protein, 11 aa, Artificial Sequence
  Description of Artificial Sequence Synthetic peptide
EEEEEEEEEEE

SEQ ID NO: 26 - protein, 12 aa, Artificial Sequence
  Description of Artificial Sequence Synthetic peptide
EEEEEEEEEEEE

SEQ ID NO: 27 - protein, 13 aa, Artificial Sequence
  Description of Artificial Sequence Synthetic peptide
EEEEEEEEEEEEE

SEQ ID NO: 28 - protein, 14 aa, Artificial Sequence
  Description of Artificial Sequence Synthetic peptide
EEEEEEEEEEEEEE

SEQ ID NO: 29 - protein, 15 aa, Artificial Sequence
  Description of Artificial Sequence Synthetic peptide
EEEEEEEEEEEEEEE

SEQ ID NO: 30 - protein, 17 aa, Artificial Sequence
  Description of Artificial Sequence Synthetic peptide
EEEEEEEEEEEEEEEEE

SEQ ID NO: 31 - protein, 18 aa, Artificial Sequence
  Description of Artificial Sequence Synthetic peptide
EEEEEEEEEEEEEEEEEE

SEQ ID NO: 32 - protein, 19 aa, Artificial Sequence
  Description of Artificial Sequence Synthetic peptide
EEEEEEEEEEEEEEEEEEE

SEQ ID NO: 33 - protein, 20 aa, Artificial Sequence
  Description of Artificial Sequence Synthetic peptide
EEEEEEEEEEEEEEEEEEEE